(19) United States
(12) Patent Application Publication
(10) Pub. No.: US 2024/0081704 A1
(43) Pub. Date: Mar. 14, 2024

(54) Introducer Housing for Blood Draw Device

(71) Applicant: Becton, Dickinson and Company, Franklin Lakes, NJ (US)

(72) Inventors: Weston F. Harding, Lehi, UT (US); Megan S. Scherich, Salt Lake City, UT (US); Yiping Ma, Layton, UT (US); Evan Barbour VandenBrink, San Mateo, CA (US)

(21) Appl. No.: 18/243,759

(22) Filed: Sep. 8, 2023

Related U.S. Application Data
(60) Provisional application No. 63/406,047, filed on Sep. 13, 2022.

(51) Int. Cl. A61B 5/15

(52) U.S. Cl. CPC A61B 5/150992; A61B 5/15003; A61B 5/150267; A61M 25/0097

(57) ABSTRACT

Provided herein is a blood draw device for use with a PIVC. The blood draw device includes a catheter, an introducer housing, and an actuator movably coupled to the introducer housing. The introducer housing includes a first member and a second member coupled together via a plurality of snaps to define an inner volume configured to movably receive the catheter. A plurality of lower snaps is formed on the first housing member and spaced apart between proximal and distal ends thereof, each of the lower snaps having a hook that is oriented downward. A first upper snap is formed on the first housing member at a distal end thereof, the first upper snap having a hook that is oriented upward. A second upper snap is formed on the second housing member at a proximal end thereof, the second upper snap having a hook that is oriented downward.

### CROSS-REFERENCE TO RELATED APPLICATION

The present application claims priority to U.S. Provisional Application Ser. No. 63/406,047 entitled “Introducer Housing for Blood Draw Device”, filed Sep. 13, 2022, the entire disclosure of which is hereby incorporated by reference in its entirety.

### BACKGROUND OF THE INVENTION

### Field of the Invention

Provided herein are devices and components for use in vascular access, and, in particular, devices and components for use with blood draw through indwelling catheters.

### Description of Related Art

Catheters are frequently utilized to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein. The catheter length may vary from a few centimeters when the VAD is a peripheral intravenous catheter (PIVC) to many centimeters when the VAD is a central venous catheter (CVC), as examples.

The catheter is commonly incorporated into a catheter adapter of the VAD to aid in the ease of use, accessibility, and utility of the catheter. A catheter adapter may be adapted to house one end of the catheter such that one end of the catheter is supported by the catheter adapter and the body and tip of the catheter extends beyond a first end of the catheter adapter. A catheter adapter generally further includes a second end adapted to receive additional components for use with the VAD. For example, the second end of a catheter adapter may include a set of threads thereon or a needleless access connector by which such components may be attached to provide access to the patient's vasculature via the attached catheter.

Recent developments in the peripheral intravenous catheter (PIVC) field have led to the emergence of technologies designed to facilitate blood draw using an in-dwelling PIVC and accompanying blood draw device. These blood draw devices have focused on the ability to reliably collect a high-quality blood sample and reduce hemolysis. The main method by which these devices work is by employing an introducer for inserting a catheter, probe, tube, or other instrument through the lumen of the catheter. A syringe or vacutainer may then be used to collect blood samples without needing to subject the patient to additional needle sticks.

The introducer of a blood draw device typically includes a housing, a catheter movable within the housing so as to be extendable out therefrom for advancement into the in-dwelling PIVC, and an advancement member that may be actuated by an operator relative to the housing, i.e., the advancement member may be moved distally by the operator to cause a corresponding movement of the catheter relative to the housing, such that advancement of the catheter out from the housing and into the in-dwelling PIVC may be selectively controlled. The housing may have a clam shell construction, whereby two halves or portions of the housing are joined together to define an inner volume in which a portion of the catheter and portion of the advancement member are received.

In use, the blood draw device is manipulated by an operator to connect the device to the in-dwelling PIVC, and to then subsequently introduce the catheter through the lumen of the PIVC for facilitating the subsequent blood draw. It is recognized that when the blood draw device is being manipulated in such a manner, the housing may experience torsional forces caused by twisting or turning of the housing by the operator. For example, torsional forces may be applied to a distal end of the end housing when a proximal end of the housing is turned/twisted relative to the distal end, and this torsional force may cause one or more snaps or connections that join the two halves of the housing together to break or become disengaged from an associated mating surface. The breaking or disengagement of one or more of these snaps or connections may result in the two halves shifting and separating from one another, thereby rendering the blood draw device unsuitable for further use. Accordingly, a need exists in the art for a blood draw device having an introducer housing that is resistant to torsional forces that might be experienced thereby during use by an operator.

### SUMMARY OF THE INVENTION

Provided herein is a blood draw device for use with a peripheral intravenous catheter (PIVC). The blood draw device includes a catheter and an introducer housing having a proximal end and a distal end, and a top surface and a bottom surface, the introducer housing having a first housing member and a second housing member each extending between the proximal end and the distal end, with the first housing member and the second housing member coupled together via a plurality of snaps formed thereon to define an inner volume configured to movably receive the catheter. The blood draw device also includes an actuator movably coupled to the introducer housing, the actuator configured to move relative to the introducer housing to move a distal end of the catheter from a position within the introducer housing to a position outside the introducer housing. The plurality of snaps includes a plurality of lower snaps formed on the first housing member and spaced apart between the proximal end and the distal end, each of the plurality of lower snaps extending out from the first housing member from a snap proximal end to a snap distal end, the snap distal end of each of the plurality of lower snaps having a hook that is oriented downward toward the bottom surface. The plurality of snaps also includes a first upper snap formed on the first housing member at the distal end, the first upper snap extending out from the first housing member from a snap proximal end to a snap distal end, the snap distal end of the first upper snap having a hook that is oriented upward toward the top surface. The plurality of snaps further includes and a second upper snap formed on the second housing member at the proximal end, the second upper snap extending out from the second housing member from a snap proximal end to a snap distal end, the snap distal end of the second upper snap having a hook that is oriented downward toward the bottom surface.

In accordance with one aspect, the first upper snap is driven further into a mating surface of the second housing member responsive to a twisting of the proximal end of the introducer housing relative to the distal end drives, to prevent separation of the first housing member from the second housing member.

In some embodiments, an alignment feature formed on the first housing member on the distal end thereof, below the first upper snap, the alignment feature engaging another mating feature on the second housing member to prevent disengaging of the first upper snap from the mating surface on the second housing member.

In some embodiments, the alignment feature is another lower snap extending out from the first housing member from a snap proximal end to a snap distal end, the snap distal end of the another lower snap having a hook that is oriented downward toward the bottom surface.

In some embodiments, the first housing member forms a portion of the top surface and the second housing member forms another portion of the top surface, the portion of the top surface separated from the another portion of the top surface to define a groove that extends generally from the proximal end to the distal end, and wherein a portion of the actuator is engaged with the groove to enable movement of the actuator relative to the introducer housing.

Another blood draw device for use with a PIVC is provided that includes a catheter and an introducer housing having a proximal end and a distal end, and a top surface and a bottom surface, the introducer housing having a first housing member and a second housing member each extending between the proximal end and the distal end, with the first housing member and the second housing member coupled together via a plurality of snaps formed thereon to define an inner volume configured to movably receive the catheter. The blood draw device also includes an actuator movably coupled to the introducer housing, the actuator configured to move relative to the introducer housing to move a distal end of the catheter from a position within the introducer housing to a position outside the introducer housing. The plurality of snaps includes a plurality of lower snaps formed on the first housing member and spaced apart between the proximal end and the distal end, each of the plurality of lower snaps extending out from the first housing member from a snap proximal end to a snap distal end, the snap distal end of each of the plurality of lower snaps having a hook that is oriented downward toward the bottom surface. The plurality of snaps also includes a first upper snap formed on the first housing member at the distal end, the first upper snap extending out from the first housing member from a snap proximal end to a snap distal end, the snap distal end of the first upper snap having a hook that is oriented toward the distal end of the introducer housing. The plurality of snaps further includes a second upper snap formed on the second housing member at the proximal end, the second upper snap extending out from the second housing member from a snap proximal end to a snap distal end, the snap distal end of the second upper snap having a hook that is oriented downward toward the bottom surface. The second housing member includes a snap stop positioned at the distal end, the snap stop extending out from the second housing member so as to be positioned proximally behind the first upper snap, adjacent to a back side of the hook thereof.

In accordance with one aspect, the snap stop prohibits proximal movement of the first upper snap relative to the second housing member, thereby preventing the first upper snap from disengaging a mating surface of the second housing member.

In some embodiments, the snap stop comprises a cap piece inserted into the second housing member.

Another blood draw device for use with a PIVC is provided that includes a catheter and an introducer housing having a proximal end and a distal end, and a top surface and a bottom surface, the introducer housing having a first housing member and a second housing member each extending between the proximal end and the distal end, with the first housing member and the second housing member coupled together via a plurality of snaps formed thereon to define an inner volume configured to movably receive the catheter. The blood draw device also includes an actuator movably coupled to the introducer housing, the actuator configured to move relative to the introducer housing to move a distal end of the catheter from a position within the introducer housing to a position outside the introducer housing. The first housing member includes a bottom joining surface extending from the proximal end to the distal end, the bottom joining surface having a plurality of detents formed therein that are spaced apart between the proximal end and the distal end. The second housing member includes another bottom joining surface that adjoins with the bottom joining surface of the first housing member, the another bottom joining surface having a plurality of bump outs formed thereon that are spaced apart between the proximal end and the distal end, so as to be aligned with the plurality of detents and interfit therewith.

In some embodiments, the plurality of snaps includes a plurality of lower snaps formed on the first housing member and spaced apart along the bottom joining surface, each of the plurality of lower snaps extending out from the first housing member from a snap proximal end to a snap distal end, the snap distal end of each of the plurality of lower snaps having a hook that is oriented downward toward the bottom surface. The plurality of snaps also includes a first upper snap formed on the first housing member at the distal end, the first upper snap extending out from the first housing member from a snap proximal end to a snap distal end, the snap distal end of the first upper snap having a hook that is oriented toward the distal end of the introducer housing. The plurality of snaps also includes a second upper snap formed on the second housing member at the proximal end, the second upper snap extending out from the second housing member from a snap proximal end to a snap distal end, the snap distal end of the second upper snap having a hook that is oriented downward toward the bottom surface.

In accordance with one aspect, interfitting of the plurality of bump outs with the plurality of detents prevents the plurality of lower snaps from disengaging mating surfaces on the second housing member when the proximal end of the introducer housing is twisted relative to the distal end, to prevent separation of the first housing member from the second housing member.

Another blood draw device for use with a PIVC is provided that includes a catheter and an introducer housing having a proximal end and a distal end, and a top surface and a bottom surface, the introducer housing having a first housing member and a second housing member each extending between the proximal end and the distal end, with the first housing member and the second housing member coupled together via a plurality of snaps formed thereon to define an inner volume configured to movably receive the catheter. The blood draw device also includes an actuator movably coupled to the introducer housing, the actuator configured to move relative to the introducer housing to move a distal end of the catheter from a position within the introducer housing to a position outside the introducer housing. The plurality of snaps includes a plurality of lower snaps formed on the first housing member and spaced apart between the proximal end and the distal end, each of the plurality of lower snaps extending out from the first housing member from a snap proximal end to a snap distal end, the snap distal end of each of the plurality of lower snaps having a hook that is oriented downward toward the bottom surface. The plurality of snaps also includes a first upper snap formed on the first housing member at the distal end, the first upper snap extending out from the first housing member from a snap proximal end to a snap distal end, the snap distal end of the first upper snap having a hook that is oriented toward the distal end of the introducer housing. The plurality of snaps further includes a second upper snap formed on the second housing member at the proximal end, the second upper snap extending out from the second housing member from a snap proximal end to a snap distal end, the snap distal end of the second upper snap having a hook that is oriented downward toward the bottom surface. The second housing member also includes a mating surface engaged by the hook of the first upper snap and a window formed in the second housing member adjacent the mating surface, with the window encircling the hook of the first upper snap being sized to restrict vertical movement of the hook of the first upper snap.

In some embodiments, a vertical gap between an edge of the window and a side surface of the hook of the first upper snap is 0.1 mm or less.

Another blood draw device for use with a PIVC is provided that includes a catheter and an introducer housing having a proximal end and a distal end, and a top surface and a bottom surface, the introducer housing having a first housing member and a second housing member each extending between the proximal end and the distal end, with the first housing member and the second housing member coupled together to define an inner volume configured to movably receive the catheter. The blood draw device also includes an actuator movably coupled to the introducer housing, the actuator configured to move relative to the introducer housing to move a distal end of the catheter from a position within the introducer housing to a position outside the introducer housing. The first housing member and the second housing member include a plurality of pegs and a plurality of peg holes joinable to secure the first housing member and the second housing member, the plurality of pegs being press fit into the plurality of peg holes.

In some embodiments, the plurality of pegs and the plurality of peg holes includes a plurality of lower peg-peg hole pairs provided on the first housing member and the second housing member and arranged so as to be spaced apart between the proximal end and the distal end, a first upper peg-peg hole pair provided on the first housing member and the second housing member at the distal end, and a second upper peg-peg hole pair provided on the first housing member and the second housing member at the proximal end.

In some embodiments, each of the plurality of pegs has a first cross-sectional shape and each of the plurality of peg holes has an opening with a second cross-sectional shape different from the first cross-sectional shape, to reduce a stress of the press fit between the pegs and the peg holes

Another blood draw device for use with a PIVC is provided that includes a catheter and an introducer housing having a proximal end and a distal end, and a top surface and a bottom surface, the introducer housing having a first housing member and a second housing member each extending between the proximal end and the distal end, with the first housing member and the second housing member coupled together via a plurality of snaps formed thereon to define an inner volume configured to movably receive the catheter. The blood draw device also includes an actuator movably coupled to the introducer housing, the actuator configured to move relative to the introducer housing to move a distal end of the catheter from a position within the introducer housing to a position outside the introducer housing. The plurality of snaps includes a plurality of lower snaps formed on the first housing member and spaced apart between the proximal end and the distal end, each of the plurality of lower snaps extending out from the first housing member from a snap proximal end to a snap distal end, the snap distal end of each of the plurality of lower snaps having a hook that is oriented downward toward the bottom surface. The plurality of snaps also includes a first upper snap formed on the first housing member at the distal end, the first upper snap extending out from the first housing member from a snap proximal end to a snap distal end, the snap distal end of the first upper snap having a hook that is oriented toward the distal end of the introducer housing. The plurality of snaps further includes a second upper snap formed on the second housing member at the proximal end, the second upper snap extending out from the second housing member from a snap proximal end to a snap distal end, the snap distal end of the second upper snap having a hook that is oriented downward toward the bottom surface. The first housing member also includes a horizontally oriented ledge extending from the proximal end to the distal end, and the second housing member further includes a stop feature positioned on an underside of the top surface, the stop feature having an abutment surface facing the horizontally oriented ledge and arranged parallel thereto, with the abutment surface separated from the horizontally oriented ledge by a distance of 0.1 mm or less.

In accordance with one aspect, the stop feature inhibits vertical movement of the first housing member relative to the second housing member responsive to a twisting of the proximal end of the introducer housing relative to the distal end, thereby preventing a corresponding proximal movement of the first upper snap off of a mating surface of the second housing member.

In some embodiments, the stop feature is a rib positioned at the proximal end and/or the distal end of the introducer housing.

### BRIEF DESCRIPTION OF THE DRAWINGS

FIG. 1 is a perspective view of a blood draw device with which embodiments of the present application may incorporated;

FIG. 2 is a top view of the blood draw device illustrated in FIG. 1;

FIG. 3 is an exploded view of the blood draw device of FIG. 1;

FIG. 4 is a perspective view of a first member of an introducer housing included in the blood draw device of FIG. 1;

FIG. 5 is a perspective view of a second member of the introducer housing included in the blood draw device of FIG. 1;

FIG. 6 is a rear perspective view of the introducer housing formed by coupling the first member illustrated in FIG. 4 to the second member illustrated in FIG. 5;

FIG. 7 is a front perspective view of the introducer housing illustrated in FIG. 6;

FIG. 8 is a cross-sectional view of the introducer taken along the line 8-8 in FIG. 6:

FIG. 9 is a rear perspective view of a lock included in the blood draw device of FIG. 1;

FIG. 10 is an exploded perspective view a catheter, a secondary catheter, and an actuator included in the blood draw device of FIG. 1;

FIG. 11 is a perspective view of the actuator illustrated in FIG. 10;

FIG. 12 is a cross-sectional view of the blood draw device taken along the line 12-12 in FIG. 2:

FIG. 13 is a side view of the blood draw device of FIG. 1 in a first configuration;

FIG. 14 is a side view of the blood draw device of FIG. 1 in a second configuration;

FIG. 15 is an exploded perspective view of a prior art introducer housing formed from a first member and a second member;

FIG. 16 is a detailed view of a portion of the introducer housing of FIG. 15, illustrating movement of a snap relative to a mating surface;

FIG. 17 is a perspective view of an introducer housing, according to an embodiment of the present application;

FIG. 18 is an exploded perspective view of the introducer housing of FIG. 17, showing a first housing member and a second housing member;

FIG. 19 is an enlarged perspective view of a snap hook portion of the introducer housing of FIG. 17;

FIG. 20 is a side view of a portion of the introducer housing of FIG. 17, showing engagement of a first upper snap with a mating surface on the second housing member,

FIG. 21 is an exploded perspective view of a portion of the introducer housing of FIG. 17, with an alignment feature added to the first housing member, according to an embodiment of the present application;

FIG. 22 is an exploded perspective view of a portion of the introducer housing of FIG. 17, with an alignment feature added to the first housing member, according to another embodiment of the present application;

FIG. 23 is an exploded perspective view of an introducer housing formed from a first member and a second member, according to an embodiment of the present application;

FIG. 24 is a side view of a portion of the introducer housing of FIG. 23, showing engagement of a first upper snap with a mating surface on the second housing member.

FIG. 25 is an exploded perspective view of an introducer housing formed from a first member and a second member, according to an embodiment of the present application;

FIG. 26 is a cross-sectional view of the introducer housing of FIG. 25 taken along line 26-26;

FIG. 27 is an exploded perspective view of an introducer housing formed from a first member and a second member, according to an embodiment of the present application;

FIG. 28 is a side view of a portion of the introducer housing of FIG. 27, showing engagement of a first upper snap with a mating surface on the second housing member.

FIG. 29 is an exploded perspective view of an introducer housing formed from a first member and a second member, according to an embodiment of the present application;

FIG. 30 is a cross-sectional view of the introducer housing of FIG. 29 taken along line 30-30;

FIG. 31 is an exploded perspective view of an introducer housing formed from a first member and a second member, according to an embodiment of the present application;

FIG. 32 is a perspective view of a pin included in the introducer housing of FIG. 31;

FIG. 33 is a perspective view of a pin hole included in the introducer housing of FIG. 31; and

FIG. 34 is a sectional view illustrating the mating of the pin and pin hole of FIGS. 32 and 33.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device (e.g., the end of the device being manipulated by the user) would be the proximal end of the device.

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

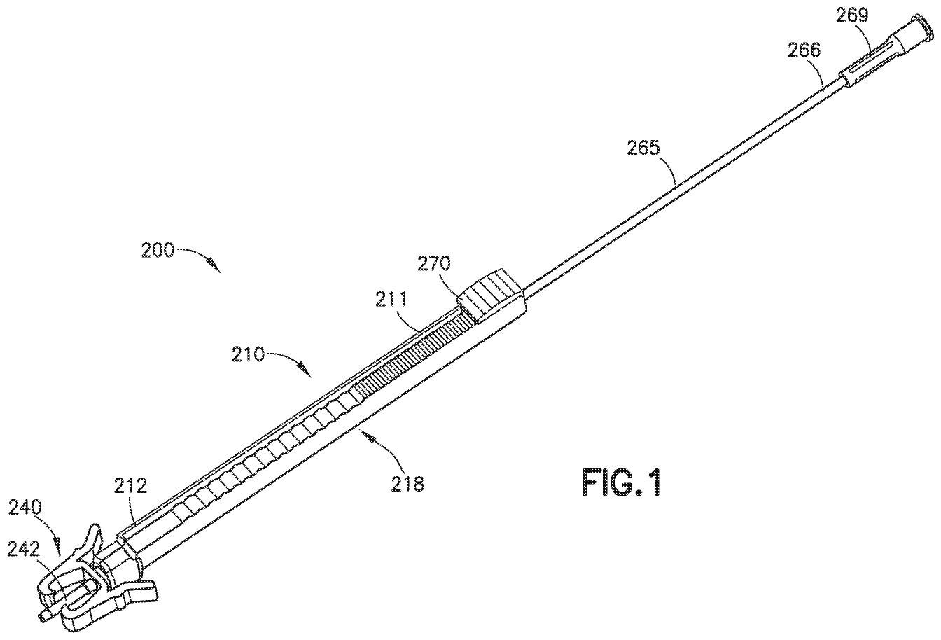

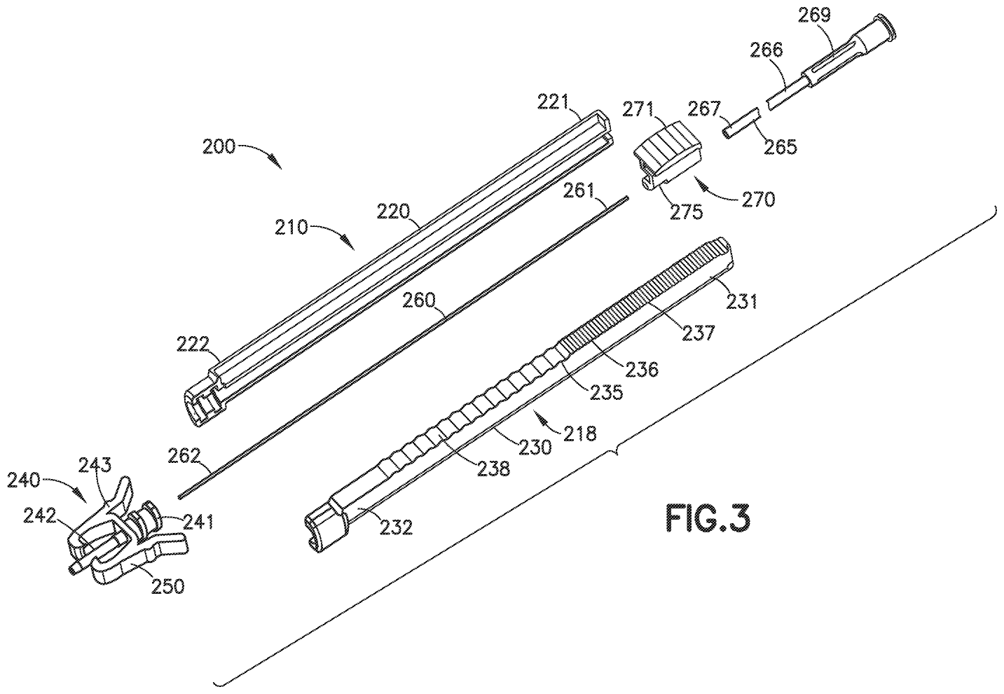

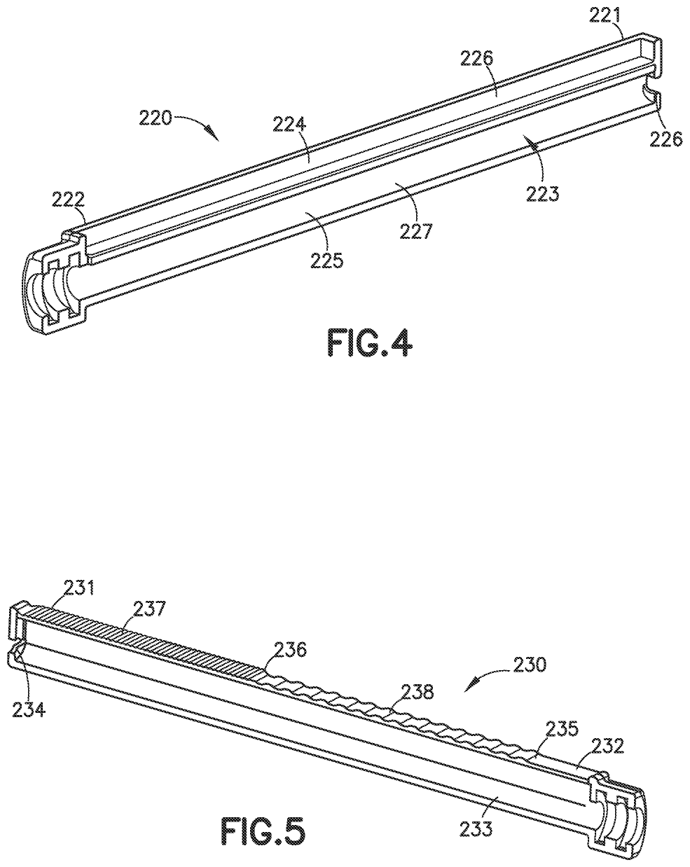

FIGS. 1-14 illustrate a blood draw device 200 (and differing configurations in which it is operated) with which embodiments of the invention may be implemented. The blood draw device 200 can be any suitable shape, size, or configuration and can be coupled to a PIVC (not shown in FIGS. 1-14), for example, via a lock and/or adapter. As described in further detail herein, a user can transition the blood draw device 200 from a first configuration to a second configuration to advance a catheter through an existing, placed, and/or indwelling PIVC (i.e., when the blood draw device 200 is coupled thereto) such that at least an end portion of the catheter is disposed in a distal position relative to the PIVC. Moreover, with peripheral intravenous lines each having a shape, size, and/or configuration that can vary based on, for example, a manufacturer of the PIVC and/or its intended usage, the blood draw device 200 can be arranged to allow the blood draw device 200 to be coupled to a PIVC having any suitable configuration and subsequently, to advance at least a portion of a catheter through the PIVC. In addition, the blood draw device 200 can be manipulated by a user to place a distal surface of the catheter a predetermined and/or desired distance beyond a distal surface of the PIVC to be disposed within a portion of a vein that receives a substantially unobstructed flow of blood.

As shown in FIGS. 1-3, the blood draw device 200 includes an introducer 210, a lock 240, a catheter 260, a secondary catheter 265, and an actuator 270. The introducer 210 can be any suitable shape, size, or configuration. For example, in some embodiments, the introducer 210 can be an elongate member having a substantially circular cross-sectional shape. In some embodiments, the shape of the introducer 210 and/or one or more features or surface finishes of at least an outer surface of the introducer 210 can be arranged to increase the ergonomics of the blood draw device 200, which in some instances, can allow a user to manipulate the blood draw device 200 with one hand (i.e., single-handed use).

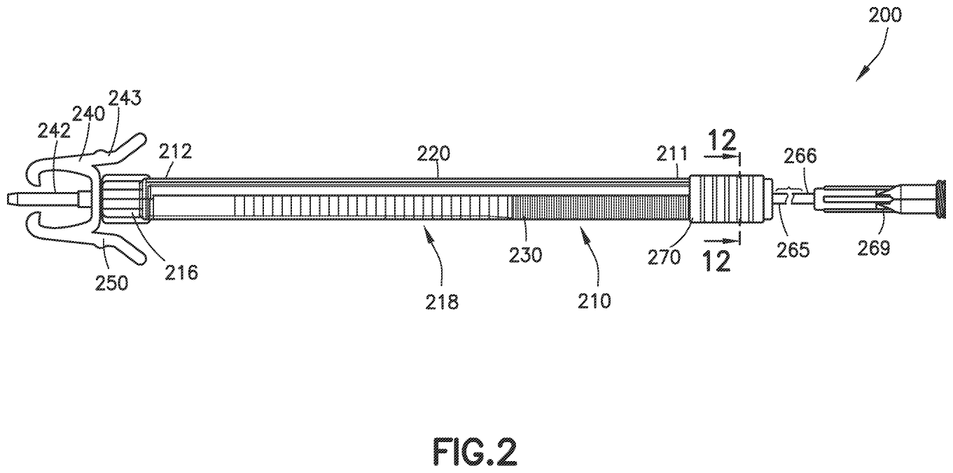

As shown in FIGS. 2-8, the introducer 210 of the blood draw device 200 includes an introducer housing 218 having a first housing member 220 and a second housing member 230 that are coupled to collectively form the housing 218. The first housing member 220 includes a proximal end portion 221, a distal end portion 222, and an inner surface 223. The inner surface 223 has a first portion 224 and a second portion 225. The proximal end portion 221 of the first housing member 220, and more specifically, a proximal wall of the first housing member 220 defines a notch 226 configured to selectively receive a portion of the secondary catheter 265, as described in further detail herein.

The second housing member 230 has a proximal end portion 231, a distal end portion 232, an inner surface 233, and a top surface 235. As described above with reference to the first housing member 220, the proximal end portion 231 of the second housing member 230, and more specifically, a proximal wall of the second housing member 230 defines a notch 234 configured to selectively receive a portion of the secondary catheter 265.

The top surface 235 of the second housing member 230 includes a set of ribs 236 distributed along a length of the second housing member 230, with each rib 236 extending along a width of the second housing member 230. The ribs 236 formed by the top surface 235 of the second housing member 230 can be any suitable shape, size, and/or configuration. For example, the set of ribs 236 includes a first portion 237 having a first size and shape, and a second portion 238 having a second size and shape, different from the first size and shape. Each rib in the second portion 238 has a size and shape that is greater than the size and shape of each rib of the first portion 237. While in the illustrated embodiment the ribs 236 are shown as being included on second housing member 230, it is recognized that ribs 236 could instead be provided on the first housing member 220, according to other embodiments.

As described in further detail herein, a portion of the actuator 270 is configured to be advanced along the top surface 235 forming the set of ribs 236 as a user moves the actuator 270 relative to the introducer housing 218, which in turn, vibrates the actuator 270 (and the catheter 260 coupled thereto). In some instances, this vibration can, for example, facilitate the advancing of the catheter 260 through a portion or the blood draw device 200, a portion of the PIVC, and/or a portion of the vasculature. In some instances, the greater size of the ribs of the second portion 238 can result in a larger amount of vibration as the actuator 270 is moved along the top surface 235 (as described above). In some instances, the greater size of the ribs of the second portion 238 can result in an increase in a force otherwise sufficient to move the portion of the actuator 270 along the top surface 235.

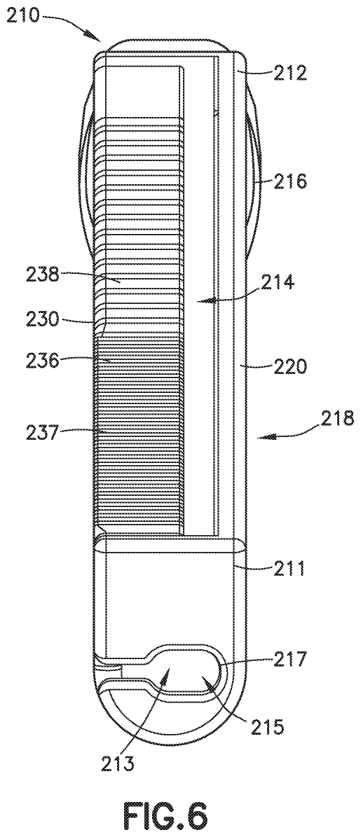

As shown in FIGS. 6-8, the first housing member 220 is configured to be coupled to the second housing member 230 to collectively form the introducer housing 218. In some embodiments, forming the introducer housing 218 from the first housing member 220 and the second housing member 230 can reduce undesirable variations in the shape and/or size of the inner surface 223 and 233 (e.g., due to draft angles and/or manufacturing tolerances) during manufacturing, which in some instances, can reduce a likelihood of kinks, bends, and/or deformations of the catheter 260 during use of the blood draw device 200. In some embodiments, forming the introducer housing 218 from the first housing member 220 and the second housing member 230 can allow at least the inner surface 223 of the first housing member 220 to form a tortuous shape that would otherwise present challenges when manufacturing the introducer housing 218 from a single work piece.

The first housing member 220 and the second housing member 230 collectively form a proximal end portion 211 and a distal end portion 212 of the introducer housing 218 and collectively define an inner volume 213 of the introducer housing 218. As shown in FIG. 6, the proximal end portion 211 of the introducer housing 218 defines an opening 217. Specifically, the opening 217 is collectively formed and/or defined by the notch 226 of the first housing member 220 and the notch 234 of the second housing member 230. The arrangement of the proximal end portion 211 is such that a portion of the opening 217 defined by the notch 226 of the first housing member 220 has a first size and/or shape and a portion of the opening 217 defined by the notch 234 of the second housing member 230 has a second size and/or shape that is less than the first size and/or shape. In other words, a portion of the opening 217 is constricted, pinched, obstructed, and/or otherwise reduced. As described in further detail herein, the opening 217 is configured to receive a portion of the secondary catheter 265, which can be moved within the opening 217 from the larger portion of the opening 217 to the reduced portion of the opening 217 (e.g., the portion formed by the notch 234 of the second housing member 230) to obstruct, pinch, and/or clamp the secondary catheter 265.

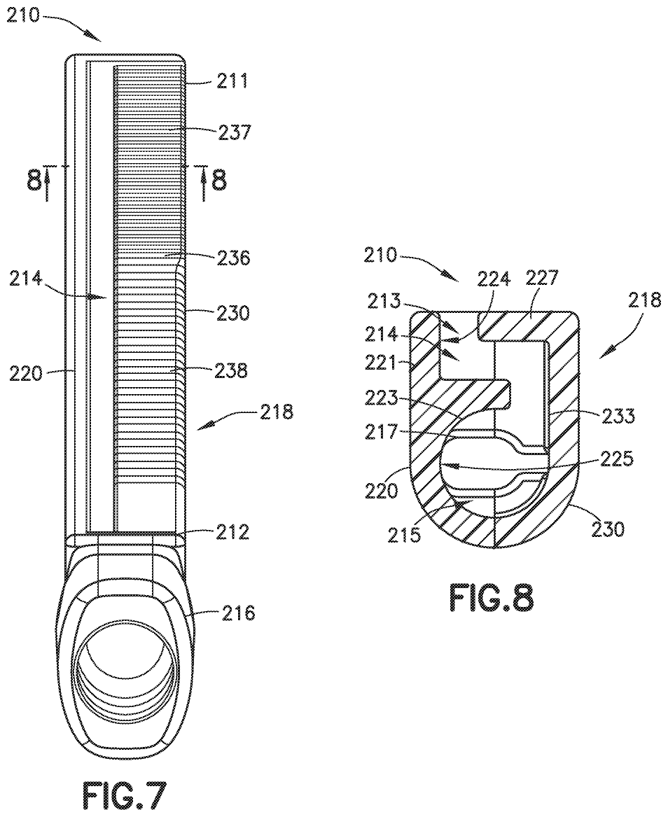

As shown in FIG. 7, the distal end portion 212 of the introducer housing 218 includes and/or otherwise forms a coupler 216. In other words, the distal end portion 222 of the first housing member 220 and the distal end portion 232 of the second housing member 230 collectively form the coupler 216 at the distal end portion 212 of the introducer housing 218. The coupler 216 can be any suitable shape, size, and/or configuration. For example, in this embodiments, the coupler 216 forms a set of threads, which can form a threaded coupling with an associated threaded portion of the lock 240, as described in further detail herein. Although not shown in FIG. 7, the distal end portion 211 of the introducer housing 218 can include and/or can be configured to receive a seal that can selectively seal and/or fluidically isolate the inner volume 213 of the introducer housing 218 (at least from an open portion of the coupler 216). In use, the seal can be transitioned from a sealed or closed configuration to an open configuration to allow, for example, a portion of the catheter 260 to pass therethrough. In some embodiments, the seal can contact an outer surface of the catheter 260 to define a seal therebetween that is operable to limit and/or substantially prevent a back flow of fluid between the outer surface of the cannula and the seal.

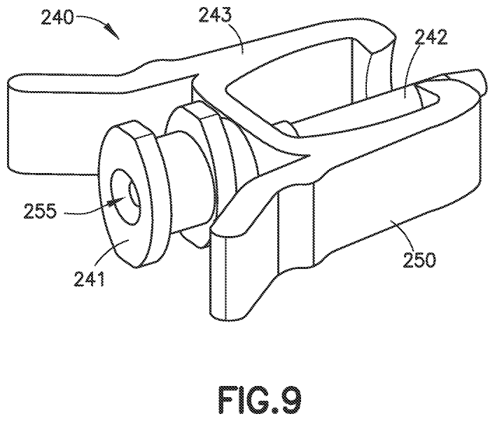

The inner surface 223 of the first housing member 220 and the inner surface 233 of the second housing member 230 collectively define the inner volume 213 of the introducer housing 218. As shown in FIG. 8, the arrangement of the inner surfaces 223 and 233 is such that the inner volume 213 has and/or defines a tortuous cross-sectional shape. For example, the inner volume 213 can have a substantially S-shaped or an at least partially S-shaped cross-sectional shape. More specifically, the inner surface 223 of the first housing member 220 includes and/or forms a ridge, tab, flange, protrusion, and/or wall configured to separate the first portion 224 of the inner surface 223 from the second portion 225 of the inner surface 223. Thus, the tortuous cross-sectional shape of the inner volume 213 forms and/or defines a first portion 214 of the inner volume 213 and a second portion 215 of the inner volume 213. In this manner, the first portion 214 of the inner volume 213 is spaced apart from the second portion 215 of the inner volume 213 without being fluidically isolated therefrom. In other words, the first portion 214 of the inner volume 213 defines an axis that is parallel to and offset from an axis defined by the second portion 215 of the inner volume 213.

As shown in FIG. 8, the first portion 214 of the inner volume 213 extend through a wall of the introducer housing 218. Similarly stated, the introducer housing 218 defines (e.g., the first housing member 220 and the second housing member 230 collectively define) a slot, channel, track, opening, and/or the like that is in fluid communication with the first portion 214 of the inner volume 213. Conversely, the second portion 215 of the inner volume 213 is entirely defined and/or enclosed (at least in the circumferential direction) by the introducer housing 218. The tortuous cross-sectional shape of the inner volume 213 is such that the second portion 215 cannot be viewed (e.g., is out of the line of sight) via the slot (in fluid communication with the first portion 214 of the inner volume 213), which in turn, can limit and/or substantially prevent contamination of the catheter 260 disposed therein.

In this embodiment, the second portion 215 of the inner volume 213 is substantially aligned with, for example, a portion of the opening 217 and a portion of an opening defined by the coupler 216. Moreover, the second portion 215 of the inner volume 213 is configured to be substantially aligned with the lock 240 when the lock is coupled to the coupler 216 of the introducer housing 218. In other words, the axis defined by the second portion 215 of the inner volume 213 is substantially co-axial with an axis defined by a portion of the lock 240, as described in further detail herein. In this manner, the second portion 215 of the inner volume 213 can movably receive, for example, a portion of the actuator 270 and a portion of the catheter 260. Thus, the actuator 270 can be moved relative to the introducer housing 218 to move the catheter 260 between a first position, in which the catheter 260 is entirely disposed within the second portion 215 of the inner volume 213, and a second position, in which at least a portion of the catheter 260 extends outside of the second portion 215 of the inner volume 213 and distal to the introducer housing 218, as described in further detail herein.

The lock 240 of the blood draw device 200 can be any suitable shape, size, and/or configuration. As described above, the lock 240 is configured to be physically and fluidically coupled to the introducer housing 218 and configured to couple the introducer 210 to the PIVC and/or any suitable intermediate device or adapter coupled to the PIVC. The lock 240 has a coupler 241, a blunt cannula 242, a first arm 243, and a second arm 250, as shown in FIG. 9. In addition, the lock 240 defines a lumen 255 extending through the coupler 241 and the blunt cannula 242. The coupler 241 is configured to couple the lock 240 to the coupler 216 of the introducer housing 218. Specifically, in this embodiment, the coupler 241 includes and/or forms one or more protrusions configured to selectively engage the threads defined and/or formed by the coupler 216 of the introducer housing 218, thereby forming a threaded coupling.

The blunt cannula 242 extends from the coupler 241 and is disposed between the first arm 243 and the second arm 250. The blunt cannula 242 can be any suitable shape, size, and/or configuration. In some embodiments, the blunt cannula 242 can have a length that is sufficient to extend through at least a portion of the PIVC or through an adapter and at least partially into or through the PIVC. Furthermore, the blunt cannula 242 can have an inner diameter (a diameter of a surface at least partially defining the lumen 255) that is similar to or slightly larger than an outer diameter of a portion of the catheter 260. Thus, the lumen 255 of the lock 240 can receive a portion of the catheter 260 when the blood draw device 200 is transitioned between the first configuration and the second configuration.

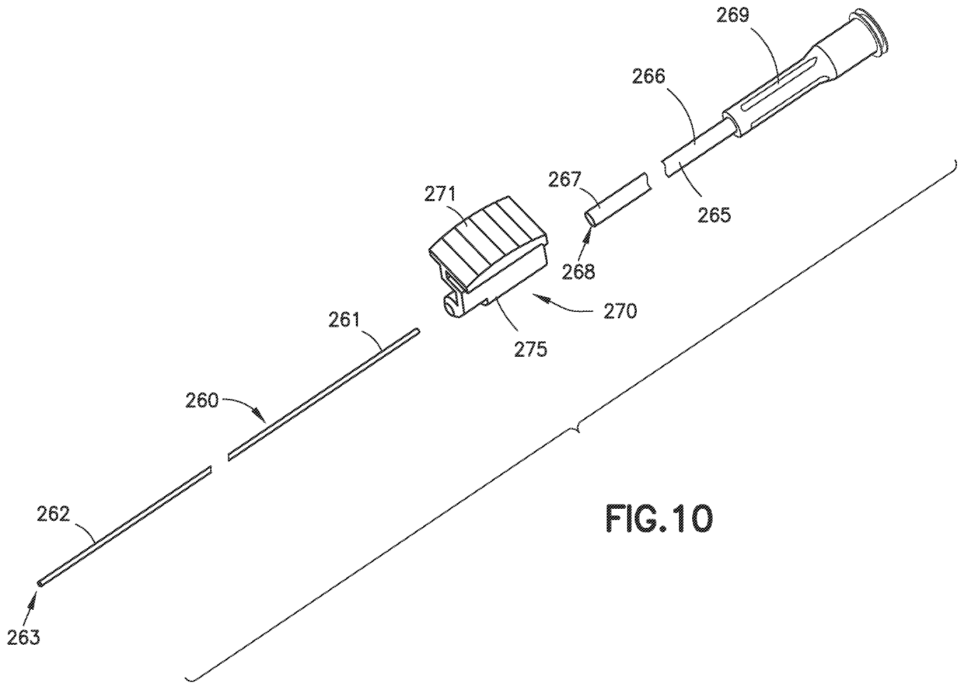

As described above, at least a portion of the catheter 260 and at least a portion of the secondary catheter 265 is movably disposed within the second portion 215 of the inner volume 213 defined by the introducer housing 218. As shown in FIG. 10, the catheter 260 has a proximal end portion 261 and a distal end portion 262 and defines a lumen 263. The proximal end portion 261 of the catheter 260 is coupled to a second portion 275 of the actuator 270. In this manner, the actuator 270 can be moved relative to the introducer housing 218 to move the catheter 260 between a first position, in which the catheter 260 is disposed within the introducer housing 218 (e.g., the entire catheter 260 is disposed within the introducer housing 218 or within the introducer housing 218 and the lock 240) and a second position, in which the distal end portion of the catheter 260 is at least partially disposed in a position distal to the lock 240 and/or the PIV (not shown) when the lock 240 is coupled to the PIV, as described in further detail herein. The distal end portion 262 can be any suitable shape, size, and/or configuration and can define at least one opening in fluid communication the lumen 263.

The secondary catheter 265 has a proximal end portion 266 and a distal end portion 267 and defines a lumen 268. A portion of the secondary catheter 265 is disposed within and extends through the opening 217 of the introducer housing 218 (e.g., collectively defined by the notches 223 and 233 of the first housing member 220 and second housing member 230, respectively). As such, the proximal end portion 266 is at least partially disposed outside of the introducer housing 218 and the distal end portion 267 is at least partially disposed within the second portion 215 of the inner volume 213 defined by the introducer housing 218. As described above, the secondary catheter 265 can be moved within the opening 217 between a first position and a second position to selectively clamp, pinch, kink, bend, and/or otherwise deform a portion of the secondary catheter 265, which in turn, obstructs, pinches, kinks, closes, seals, etc. the lumen 268 of the secondary catheter 265. For example, the first position can be associated and/or aligned with a first portion of the opening 217 having a larger perimeter and/or diameter than a perimeter and/or diameter of a second portion of the opening 217 associated and/or aligned with the second position. Thus, a user can manipulate the secondary catheter 265 to occlude the lumen 268 of the secondary catheter 265, thereby limiting, restricting, and/or substantially preventing a flow of a fluid therethrough.

As shown in FIG. 10, the proximal end portion 266 of the secondary catheter 265 is coupled to and/or otherwise includes a coupler 269. The coupler 269 is configured to physically and fluidically couple the secondary catheter 265 to any suitable device such as, for example, a fluid reservoir, fluid source, syringe, evacuated container holder (e.g., having a sheathed needle or configured to be coupled to a sheathed needle), pump, and/or the like. The distal end portion 267 of the secondary catheter 265 is at least partially disposed within the second portion 215 of the inner volume 213 defined by the introducer housing 218 and is coupled to a second portion 275 of the actuator 270. In some embodiments, the secondary catheter 265 can have a larger diameter than the catheter 260 such that the proximal end portion 261 of the catheter 260 is at least partially disposed within the lumen 268 defined by the secondary catheter 265 when the catheter 260 and the secondary catheter 265 are coupled to the second portion 275 of the actuator 270. In some embodiments, such an arrangement can, for example, reduce and/or substantially prevent leaks associated with fluid flowing between the catheter 260 and the secondary catheter 265. In some embodiments, such an arrangement can also limit, reduce, and/or substantially prevent hemolysis of a volume of blood as the volume of blood flows through the catheter 260 and the secondary catheter 265. In this manner, when the coupler 269 is coupled to a fluid reservoir, fluid source, syringe, evacuated container, pump, etc., the secondary catheter 265 establishes fluid communication between the reservoir, source, pump, etc. and the catheter 260.

The actuator 270 of the blood draw device 200 is coupled to the catheter 260 and can be moved along a length of the introducer housing 218 to transition the blood draw device 200 between its first configuration, in which the catheter 260 is in the first position, and its second configuration, in which the catheter 260 is in the second position. The actuator 270 can be any suitable shape, size, and/or configuration. For example, in some embodiments, the actuator 270 can have a size and shape that is associated with and/or based at least in part on a size and/or shape of the introducer housing 218.

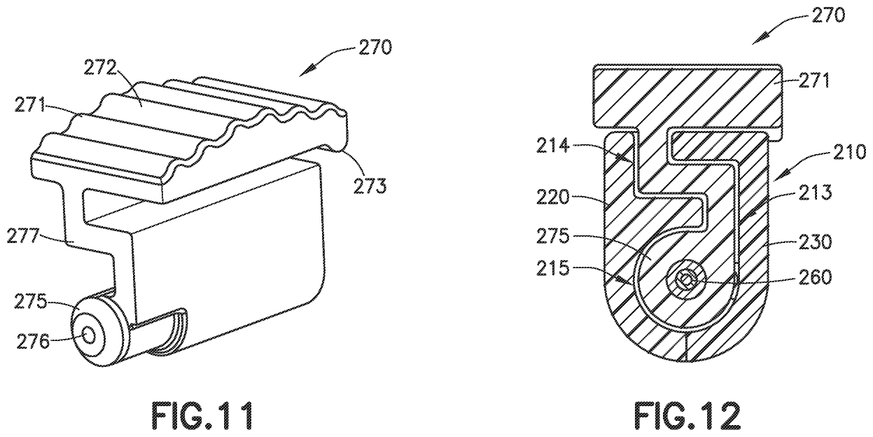

As shown in FIGS. 11 and 12, the actuator 270 includes a first portion 271, the second portion 275, and a wall 277 extending therebetween. The first portion 271 of the actuator 270 is at least partially disposed within the first portion 214 of the inner volume 213 defined by the introducer housing 218 and the second portion 275 of the actuator 270 is disposed within the second portion 215 of the inner volume 213, as described above. The first portion 271 of the actuator 270 includes an engagement member 272. The arrangement of the actuator 270 is such that the engagement member 272 is disposed outside of the introducer housing 218 while the rest of the first portion 271 is within the first portion 214 of the inner volume 213 defined by the introducer housing 218. As such, the engagement member 272 can be engaged and/or manipulated by a user (e.g., by a finger or thumb of the user) to move the actuator 270 relative to the introducer housing 218. In some embodiments, the engagement member 272 can include a set of ridges and/or any suitable surface finish that can, for example, increase the ergonomics of the actuator 270 and/or blood draw device 200.

The engagement member 272 includes a tab 273 that, in the illustrated embodiment, is disposed at or near a proximal end portion of the engagement member 272, although it is recognized that the tab 273 could be near the distal end, or anywhere between the proximal and distal ends. The tab 273 can be any suitable tab, rail, ridge, bump, protrusion, knob, roller, slider, etc. that extends from a surface of the engagement member 272. The tab 273 is configured to selectively engage the top surface 235 of the second housing member 230 of the introducer housing 218. More specifically, the tab 273 is in contact with the ribs 236 formed by the second housing member 230 and moves along each successive rib as the actuator 270 is moved along a length of the introducer housing 218, with interaction of the tab 273 with the ribs 236 causing vibration of the actuator 270.

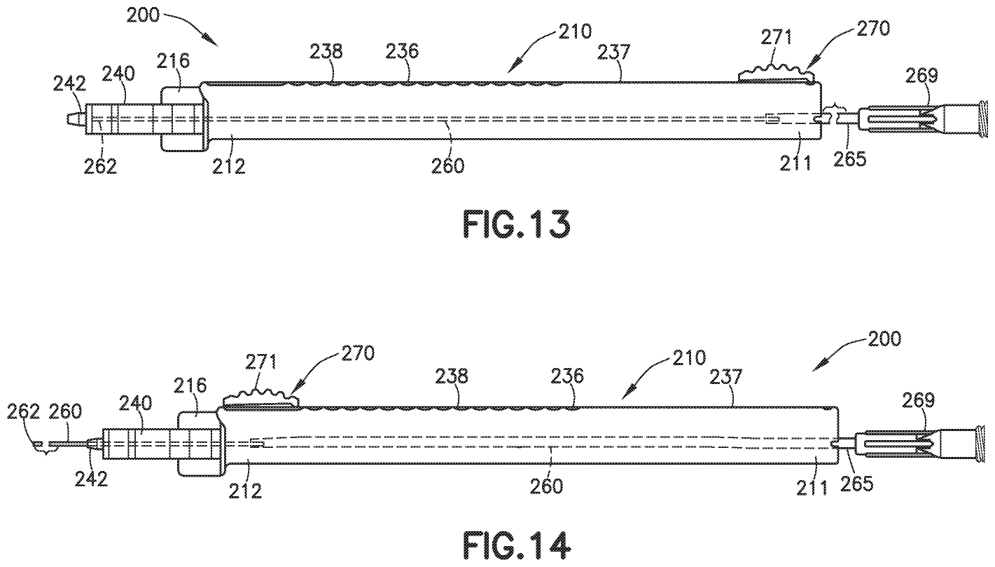

Referring now to FIGS. 13 and 14, changing of the blood draw device 200 between the first configuration and second configuration is shown. The blood draw device 200 can be in the first configuration prior to use and can be transitioned by a user (e.g., a doctor, physician, nurse, technician, phlebotomist, and/or the like) from the first configuration (FIG. 13) to the second configuration (FIG. 14) to dispose at least the distal end portion 262 of the catheter 260 in a distal position relative to the introducer housing 218 (e.g., within an indwelling PIV (not shown) or distal to the indwelling PIV).

The blood draw device 200 is in the first configuration of FIG. 13 when the catheter 260 is disposed in the first position within the introducer housing 218. In some embodiments, substantially the entire catheter 260 is disposed within the housing when the catheter 260 is in the first position. In such embodiments, the introducer housing 218 can include the seal or the like (as described above) that can substantially seal the distal end portion 212 of the housing 210 to isolate the catheter 260 within the second portion 215 of the inner volume 213. In other embodiments, the catheter 260 is disposed within the housing 210 and the lock 240 when catheter 260 is in the first position.

The actuator 270 is disposed in a proximal position when the blood draw device 200 is in the first configuration, and the user may engage the engagement member 272 of the first portion 271 of the actuator 270 to move the actuator 270 relative to the introducer housing 218, which in turn, moves the catheter 260 from the first position (e.g., disposed within the introducer housing 218) toward the second position. In this manner, the catheter 260 is moved through the second portion 215 of the inner volume 213 and the lumen 255 of the lock 240 and, as such, at least the distal end portion 262 of the catheter 260 is disposed outside of and distal to the lock 240.

The blood draw device 200 is in the second configuration of FIG. 14 when the catheter 260 is disposed in the second position. The second position of the catheter 260 is reached when the distal end portion 262 of the catheter 260 is placed in a desired position relative to a distal end portion of the PIVC. In some instances, for example, a distal end of the catheter 260 can be substantially flush with a distal end of the PIVC when the catheter 260 is in the second position. In other instances, the distal end of the catheter 260 can extend a predetermined distance beyond the distal end of the PIVC (e.g., distal to the distal end of the PIVC), such that the distal surface of the catheter 260 is positioned within the vein at a predetermined distance beyond the distal surface of the catheter 260 (e.g., a position within a vein that is substantially free from debris (e.g., fibrin/blood clots) otherwise surrounding the distal end portion of the PIVC).

With the catheter 260 in the second position (e.g., with the blood draw device 200 in the second configuration shown, for example, in FIG. 14), the user can establish fluid communication between a fluid reservoir, fluid source, syringe, and/or the like and the catheter 260. With the catheter 260 in fluid communication with the fluid reservoir and/or fluid source, the blood draw device 200 can then aspirate a volume of blood from the vein based at least in part on disposing the distal surface of the catheter 260 at the predetermined and/or desired distance beyond the distal surface of the PIVC. In some instances, once a desired amount of blood has been collected and/or once a desired volume of a drug has been delivered to the patient, the user can move the actuator 270 in the proximal direction, such as moving the actuator 270 back to its proximal most position, so as to move the catheter 260 back to the first position.

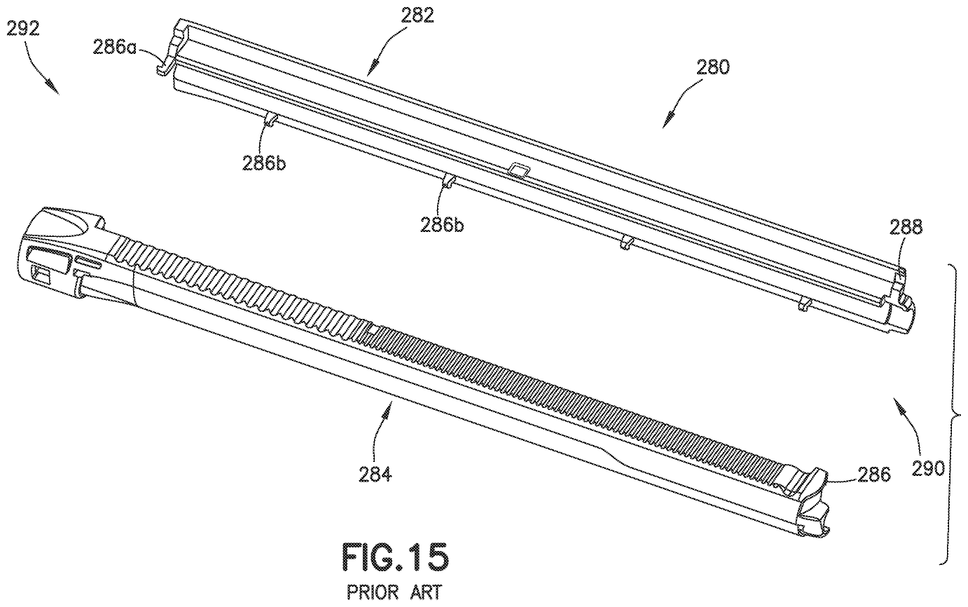

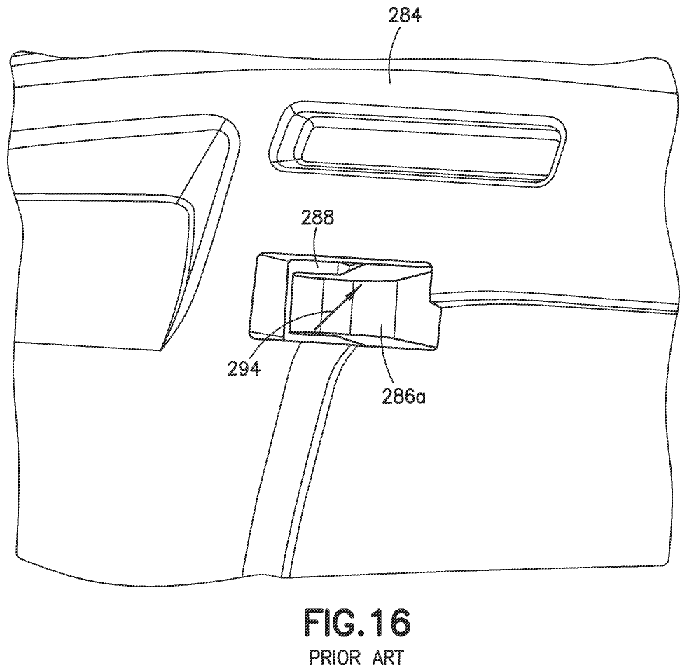

Referring now to FIG. 15, a known prior art introducer housing 280 is provided that may be utilized as the introducer housing for the blood draw device 200 of FIGS. 1-14. The introducer housing 280 includes engagement features that secure a first housing member 282 to a second housing member 284. Specifically, a plurality of cantilevered snaps 286 (hereafter “snaps”) and associated mating surfaces 288 (e.g., flat surfaces or notches) are provided on the first and second housing members 282, 284 that interact with one another to secure the first housing member 282 to the second housing member 284, with a hooked end portion of each of the cantilevered snaps 286 forming a snap-fit engagement with its associated mating surface 288.

In operation of the blood draw device of FIGS. 1-14, it is recognized that the introducer housing 218 may be manipulated by an operator, such as when connecting the blood draw device 200 to a PIVC and when subsequently advancing the actuator along 270 the introducer housing 218 to introduce the catheter 260 through the lumen of the PIVC catheter for facilitating the subsequent blood draw. During such manipulation, the introducer housing 218 may experience torsional forces caused by twisting or turning of the introducer housing 218 by the operator, such as when the proximal end 211 of the housing is turned/twisted relative to the distal end 212, and this torsional force may cause the first and second members 220, 230 to become disengaged. For example, with the introducer housing 280 of FIG. 15, twisting of a proximal end 290 of the housing relative to a distal end 292 may cause a distally positioned snap (snap 286a) to break or become disengaged—with disengagement of the snap 286a occurring due to a distally oriented hook portion of the snap 286a shifting upward and proximally off its mating surface 288 responsive to the twisting of the housing proximal end 290, as shown in FIG. 16 by arrow 294. The breaking/disengaging of the distally positioned snap 286a allows for a sideways shifting between the first and second members 284, 286 that results in lower snaps 286b becoming disengaged, such that the first housing member 284 becomes separated from the second housing member 286.

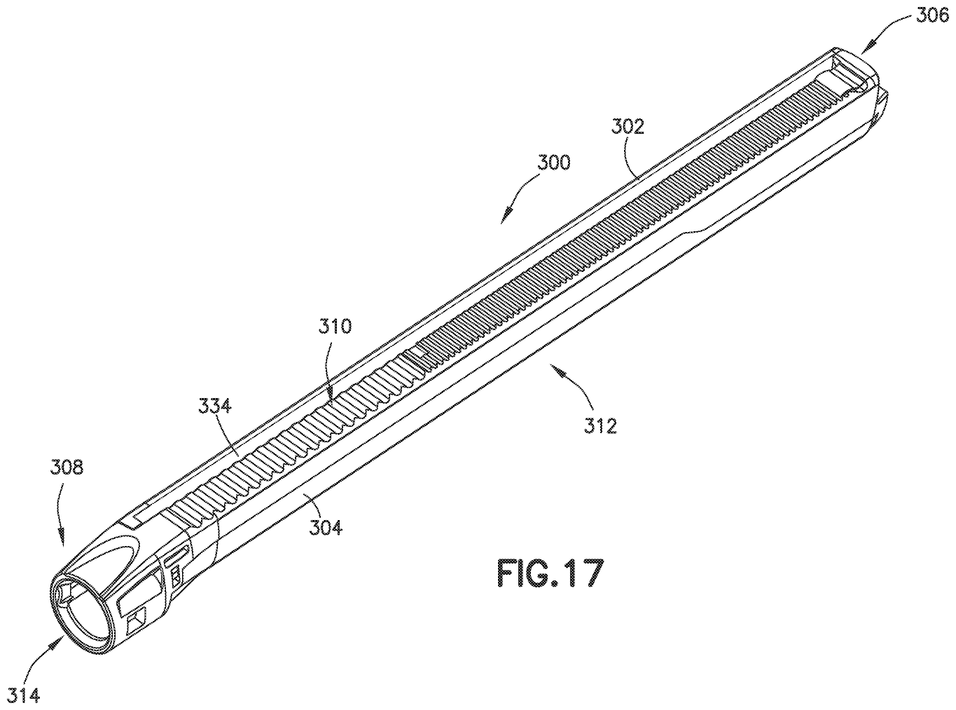

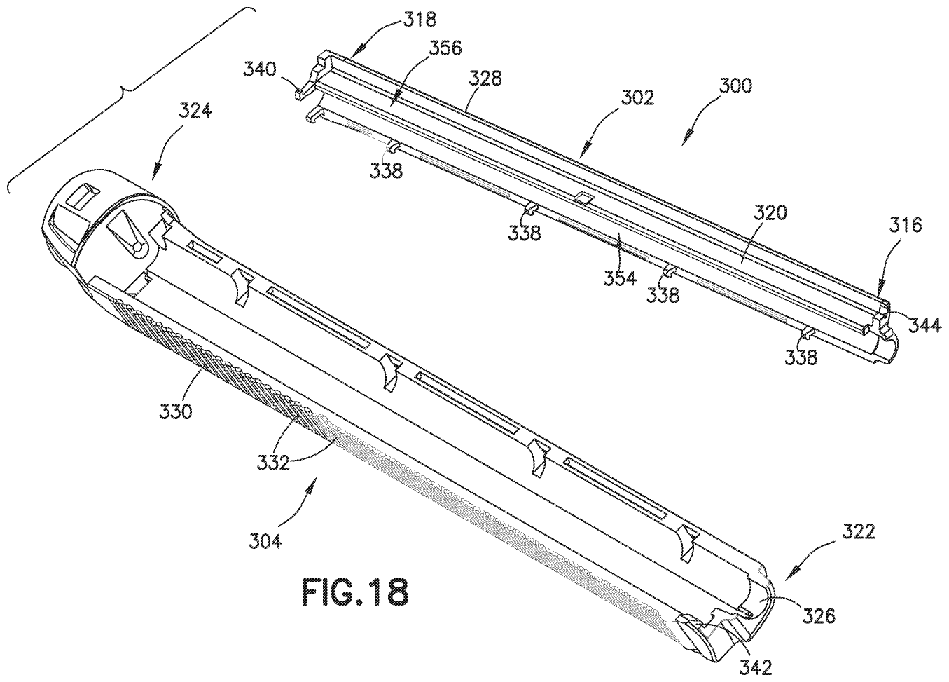

FIGS. 17-34 illustrate various embodiments of introducer housings that may be implemented with the blood draw device of FIGS. 1-14, and which provide an improvement over the prior art introducer housing shown in FIG. 15. In particular, the introducer housings shown in FIGS. 17-34 are resistant to torsional forces applied thereto, so as to provide a robust introducer housing that prevents breaking and/or separation thereof during use. The various embodiments of the introducer housings shown in FIGS. 17-34 share a plurality of common components or features, and thus common numbering is used to identify identical components/features in the introducer housings and/or common orientations (e.g., top/bottom surfaces and proximal/distal ends), but the introducer housings include differing components and features that counteract torsional forces applied to the introducer housing and prevent separation of the first and second housing members.

Referring now to FIGS. 17-22, an introducer housing 300 is provided that is resistant to torsional forces applied thereto, so as to provide a robust introducer housing 300 that prevents breaking and/or separation thereof during use. The introducer housing 300 includes a first housing member 302 and a second housing member 304 that each extend lengthwise between a proximal end 306 and a distal end 308 of the introducer housing 300. When joined together, the first and second housing members 302, 304 form a top surface 310 and a bottom surface 312 of the introducer housing 300 and define an inner volume 314.

First housing member 302 includes a proximal end 316 and a distal end 318, and an inner surface 320, and second housing member 304 likewise includes a proximal end 322, a distal end 324, and an inner surface 326. A top surface 328 of the first housing member 302 and a top surface 330 of the second housing member 304 collectively define the top surface 310 of the introducer housing 300. The top surface 328 of the first housing member 302 generally forms a ridge extending a length of the first housing member 302. The top surface 330 of the second housing member 304 includes a set of ribs 332 distributed along a length of the second housing member 304, with each rib 332 extending along a width of the top surface 330 of the second housing member 304 and successively distributed along the length thereof. As previously described, a portion of the actuator 270 of the blood draw device 200 (see, FIGS. 1-14) may be advanced along the top surface 310 of the introducer housing 300 as a user moves the actuator 270 relative to the introducer housing 300. The actuator 270 is advanced along the set of ribs 332 formed on top surface 330 of the second housing member 304 and also advances along or through a lengthwise gap 334 present between the top surface 328 of the first housing member 302 and the top surface 330 of the second housing member 304, with the gap 334 serving as a groove or track within which a portion of the actuator 270 is seated.

For joining the first housing member 302 and the second housing member 304 together, a plurality of cantilevered snaps 338, 340, 342 (hereafter “snaps”) are provided on the members. As will be described below, snaps 338, 340, 342 are formed on the first and second housing members 302, 304 and extend out therefrom to engage corresponding mating surfaces 344 on the opposite member to secure the first housing member 302 and the second housing member 304 together.

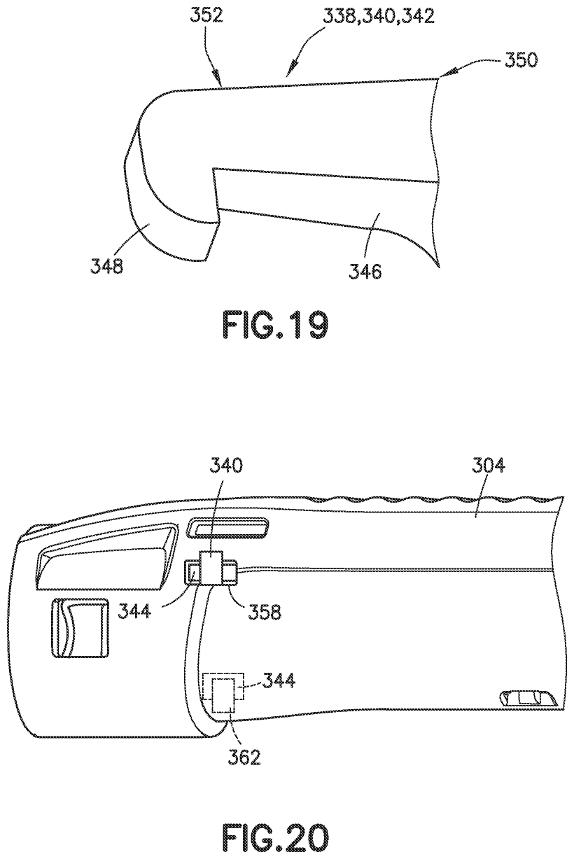

A more detailed view of the structure of the snaps 338, 340, 342 is provided in FIG. 19. Each snap 338, 340, 342 generally includes a beam member 346 and a hook portion 348. A proximal end 350 of the beam member 346 is joined to the respective first housing member 302 or second housing member 304 with which the snap is integrally formed. A distal end 352 of the beam member 346 includes the hook portion 348 formed thereon, with the hook portion 348 extending orthogonally away from the beam member 346. An inner surface of the hook portion 348 may clip over a mating surface of the opposite member 302, 304 of the introducer housing 300, to thereby secure the members together.

According to an embodiment, a plurality of snaps are provided on the first housing member 302 that are generally categorized as lower snaps 338 and a (first) upper snap 340. The lower snaps 338 are formed on a lower portion 354 of the inner surface 320 of first housing member 302 and are aligned lengthwise along the lower portion 354 between the proximal end 316 and the distal end 318. The lower snaps 338 may be spaced apart equidistant from one another along the lower portion 354, to provide a desirable arrangement of engagement points between the first housing member 302 and second housing member 304 along a majority of the length of the transducer housing 300. The upper snap 340 is formed on an upper portion 356 of the inner surface 320 of first housing member 302 and is positioned at the distal end 318 of the first housing member 302, to provide an engagement point between the first housing member 302 and second housing member 304 at the distal end 308 of the introducer housing 300. Each of the lower snaps 338 is constructed such that the hook portion 348 thereof is oriented downward toward the bottom surface 312 of introducer housing 300, while the upper snap 340 is constructed such that the hook portion 348 thereof is oriented upward toward the top surface 310 of introducer housing 300. The lower snaps 338 and upper snap 340 of the rust housing member 302 each engage a respective mating surface 344 provided on the second housing member 304, which may be formed as a notch or a flat surface. As best seen in FIG. 20, some mating surfaces 344 provided on second housing member 304 may be positioned within or adjacent to a window 358 formed in the second housing member 304 that accommodates the hook portion 348 therein.

A snap is also provided on the second housing member 304 that is categorized as a (second) upper snap 342. The upper snap 342 is formed on an upper portion of the inner surface 326 of second housing member 304 and is positioned at the proximal end 322 of the second housing member 304, to provide an engagement point between the first housing member 302 and second housing member 304 at the proximal end 306 of the introducer housing 300. The upper snap 342 is constructed such that the hook portion 348 thereof is oriented downward toward the bottom surface 312 of introducer housing 300. The upper snap 342 of the second housing member 304 engages a respective mating surface 344 provided on the first housing member 302, which is formed as a notch formed in the first housing member 302 that accommodates the hook portion 348 therein.

When one or more regions of the introducer housing 300 is exposed to a twisting motion, such as a clockwise twisting of the proximal end 306 while the distal end 308 remains held straight, torsional force is applied to the snaps 338, 340, 342 that may stress the snaps (i.e., stress the hook portion 348 and/or beam section 346) and urge the snaps toward separating from their respective mating surface 344. Specifically, application of a clockwise twisting motion to the proximal end 306 of introducer housing 300 may urge the downward oriented hook portion 348 of lower snaps 338 upward, which could cause disengagement of the hook portion 348 from its mating surface 344 on second housing member 304. However, the upward oriented hook portion 348 of the upper snap 340 on first housing member 302 counteracts any upward movement of the hook portion 348 of lower snaps 338, as application of a clockwise twisting motion to the proximal end 306 of introducer housing 300 drives the upward oriented hook portion 348 of upper snap 340 further into engagement with its respective mating surface 344 on second housing member 304, as best seen in FIG. 20. Additionally, the downward oriented hook portion 348 of the upper snap 342 on second housing member 304 counteracts any upward movement of the hook portion 348 of lower snaps 338, as application of a clockwise twisting motion to the proximal end 306 of introducer housing 300 drives the downward oriented hook portion 348 of upper snap 342 further into engagement with its respective mating surface 344 on first housing member 302. The driving of the upward oriented hook portion 348 of upper snap 340 further into engagement with its respective mating surface 344 on first housing member 302, at the distal end 308 of introducer housing 300, and the driving of the downward oriented hook portion 348 of upper snap 342 further into engagement with its respective mating surface 344 on first housing member 302, at the proximal end 306 of introducer housing 300, thus maintains engagement between the first housing member 302 and the second housing member 304 when the introducer housing 300 is exposed to a twisting motion.

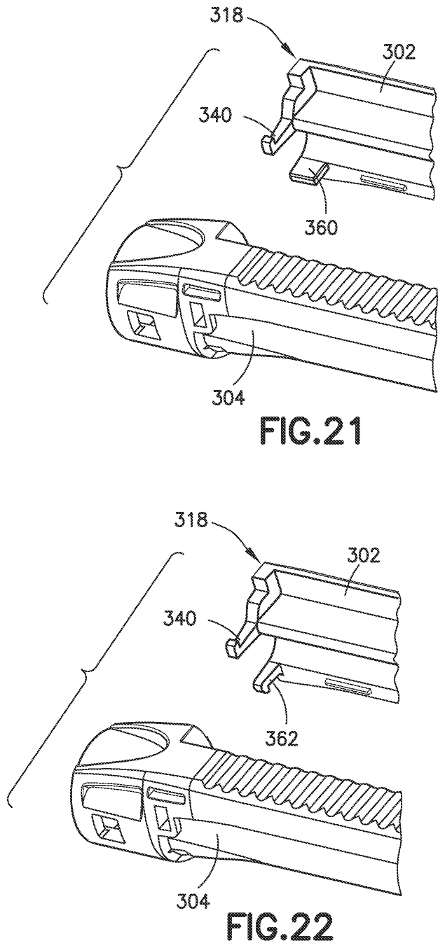

Referring now to FIGS. 21 and 22, to further ensure continued engagement between the first housing member 302 and the second housing member 304 when the introducer housing 200 is exposed to a twisting motion, an additional alignment feature 360, 362 may be formed on the first housing member 302 at the distal end 318 thereof. The alignment feature 360, 362 is formed on the lower portion 354 of the inner surface 320 of first housing member 302 at a location below upper snap 340 (i.e., closer to the bottom surface 312 of introducer housing 300) and protrudes out from first housing member 302 toward second housing member 304 to engage with a mating feature 344 thereon. In the embodiment of FIG. 21, the alignment feature is configured as a flat tab member 360 that may engage a corresponding opening in the second housing member 304. In the embodiment of FIG. 22, the alignment feature is configured as a cantilevered snap 362 configured and oriented the same as lower snaps 338—i.e., with a hook portion 348 of the snap 362 oriented downward toward the bottom surface 312 of introducer housing 300 and engageable with a respective mating surface 344 (i.e., notch or flat surface) provided on the second housing member 304, as shown in phantom in FIG. 20. Inclusion of the additional alignment feature 360, 362 at the distal end 318 of first housing member 302 provides a further means of inhibiting disengagement of the upper snap 340 from the mating surface 344 on second housing member 304.

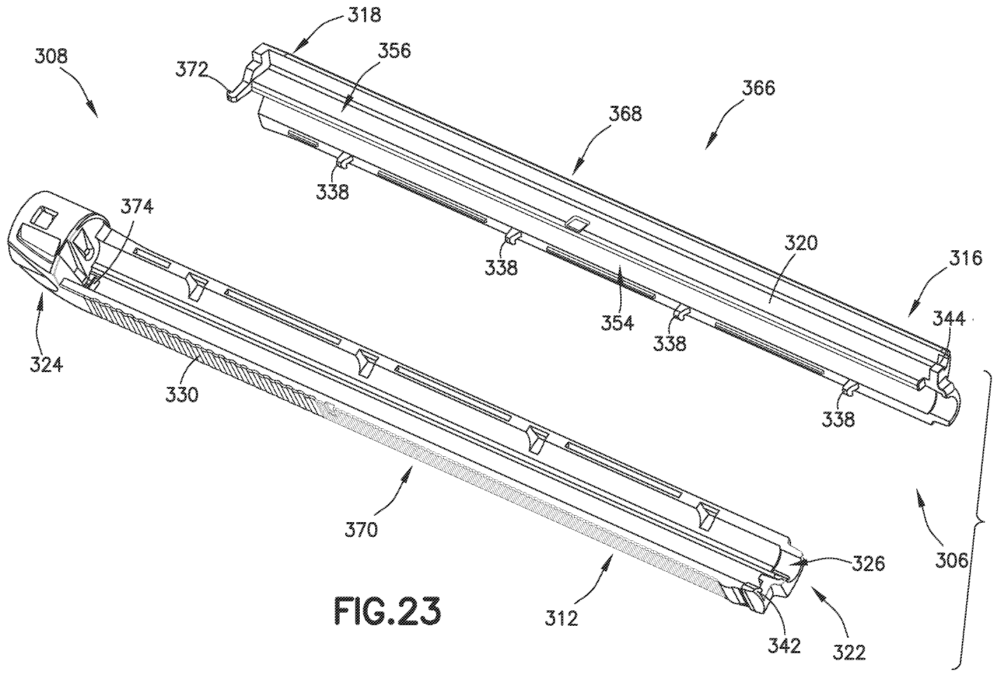

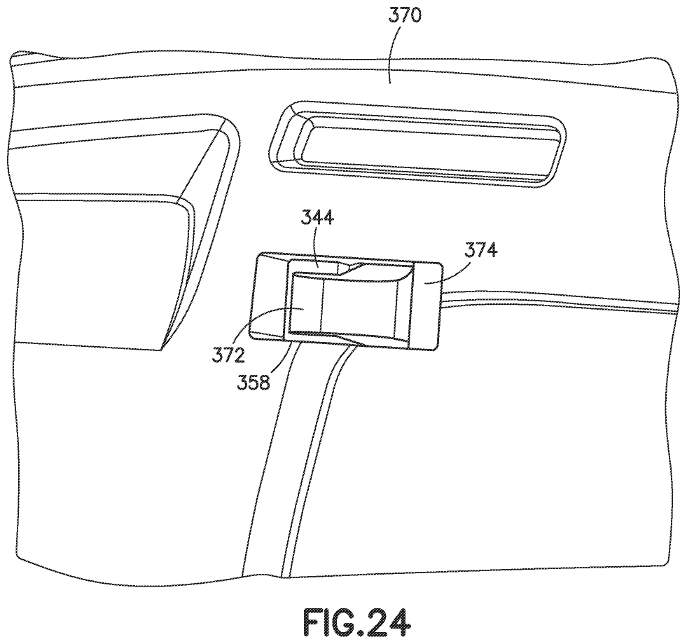

Referring now to FIGS. 23 and 24, another embodiment of an introducer housing 366 is provided that is resistant to torsional forces applied thereto. The construction of the introducer housing 366 aligns closely with that of the introducer housing 300 of FIGS. 17-22, but a different snap configuration and additional stop features are provided in the introducer housing 366 to counteract torsional forces applied to the introducer housing 366 and prevent separation of the first and second housing members 368, 370 thereof.

As shown in FIG. 23, introducer housing 366 includes lower snaps 338 that are formed on a lower portion 354 of the inner surface 320 of first housing member 368 and are aligned lengthwise along the lower portion 354 between the proximal end 316 and the distal end 318. The lower snaps 338 may be spaced apart equidistant from one another along the lower portion 354, to provide a desirable arrangement of engagement points between the first housing member 368 and second housing member 370 along a majority of the length of the transducer housing 366. A (first) upper snap 372 is formed on an upper portion of the inner surface 320 of first housing member 368 and is positioned at the distal end 318 of the first housing member 368, to provide an engagement point between the first housing member 368 and second housing member 370 at the distal end 308 of the introducer housing 366. Each of the lower snaps 338 is constructed such that the hook portion 348 thereof is oriented downward toward the bottom surface 312 of introducer housing 366, while the upper snap 372 is constructed such that the hook portion 348 thereof is oriented toward the distal end 308 of the introducer housing 366. The lower snaps 338 and upper snap 372 of the first housing member 368 each engage a respective mating surface 344 provided on the second housing member 370, which may be formed as a notch or a flat surface. Some mating surfaces 344 provided on second housing member 370 may be positioned within or adjacent to a window 358 formed in the second housing member 370 that accommodates the hook portion 348 therein.

A snap is also provided on the second housing member 370 that is categorized as a (second) upper snap 342. The upper snap 342 is formed on an upper portion of the inner surface 326 of second housing member 370 and is positioned at the proximal end 322 of the second housing member 370, to provide an engagement point between the first housing member 368 and second housing member 370 at the proximal end 306 of the introducer housing 366. The upper snap 342 is constructed such that the hook portion 348 thereof is oriented downward toward the bottom surface 312 of introducer housing 366. The upper snap 342 of the second housing member 370 engages a respective mating surface 344 provided on the first housing member 368, which may be formed as a notch.

Also provided at the distal end 324 of the second housing member 370 is a snap stop 374, as seen in FIG. 24. The snap stop 374 is formed adjacent the mating surface 344 and window 355 of second housing member 370 that receive the upper snap 372, and is positioned on second housing member 370 so as to be proximally behind the upper snap 372 of first housing member 368. That is, with the hook portion 348 of upper snap 372 oriented toward the distal end 308 of introducer housing 366, the snap stop 374 is positioned on second housing member 370 so as to be behind the hook portion 348. In some embodiments, the snap stop 374 may be provided as a cap piece that is inserted into a retaining feature of the second housing member 370, while in other embodiments the snap stop 374 may be integrally formed as part of the second housing member 370.

When one or more regions of the introducer housing 366 is exposed to a twisting motion, such as a clockwise twisting of the proximal end 306 while the distal end 308 remains held straight, torsional force is applied to the snaps 338, 372, 342 that may stress the snaps (i.e., stress the hook portion 348 and/or beam sections) and urge the snaps toward separating from their respective mating surface 344. Specifically, application of a clockwise twisting motion to the proximal end 306 of introducer housing 366 may urge the downward oriented hook portion 348 of lower snaps 338 upward and the distally oriented hook portion 348 of upper snap 372 upward and proximally forward, which could cause disengagement of the upper snap 372 from its mating surface 344 on second housing member 370.

However, the positioning of snap stop 374 proximally behind the hook portion 348 of upper snap 372 prohibits proximal movement of the upper snap 372 relative to its mating surface 344 on second housing member 370, thereby preventing the upper snap 372 from disengaging a mating surface 344 of the second housing member 370. That is, any proximal movement of the distally oriented hook portion 348 of upper snap 372 that might occur responsive to twisting of the proximal end 306 of introducer housing 366 is prevented by the snap stop 374, as the backside of hook portion 348 bumps up against snap stop 374 after only a minimal amount of proximal movement of the upper snap 372 relative to its mating surface 344. Accordingly, engagement between the first housing member 368 and the second housing member 370 is maintained when the introducer housing 366 is exposed to a twisting motion.

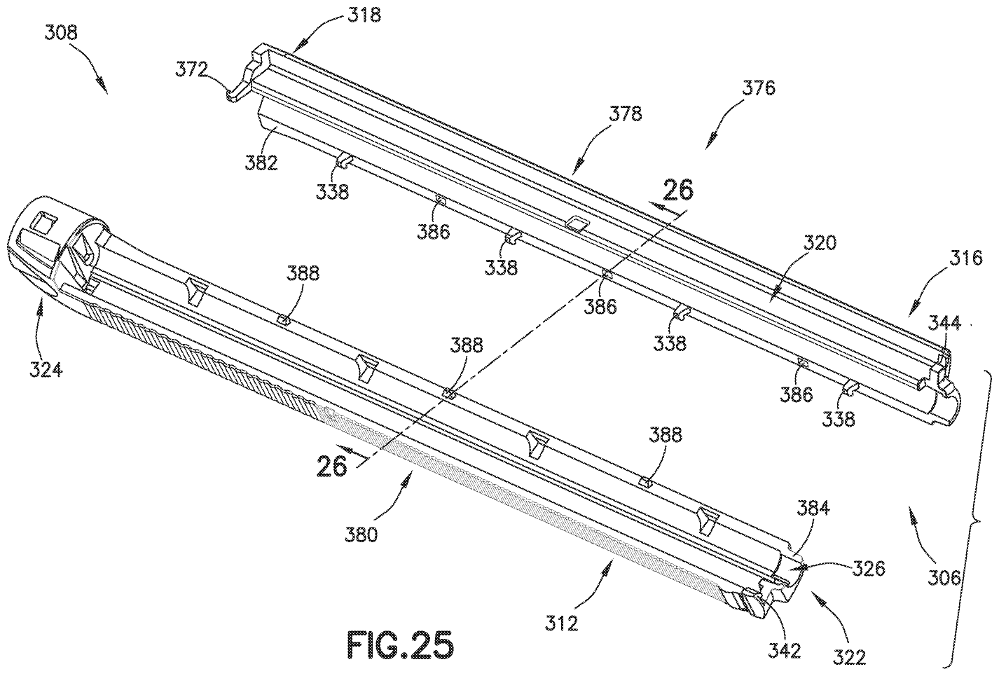

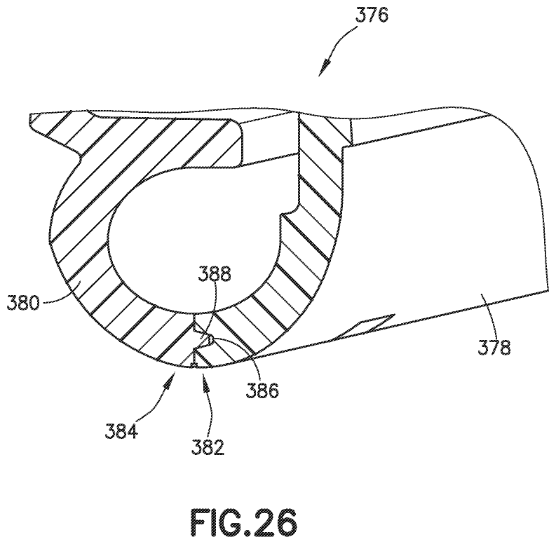

Referring now to FIGS. 25 and 26, another embodiment of an introducer housing 376 is provided that is resistant to torsional forces applied thereto. The arrangement and configurations of snaps on the introducer housing 376 of FIGS. 25 and 26 is identical to that of the introducer housing 366 of FIGS. 23 and 24 (i.e., lower snaps 338 and a second upper snap 342 with downward oriented hook portions 348 and a first upper snap 372 with a distally oriented hook portion 348), but additional alignment and mating features are provided in the introducer housing 376 to counteract torsional forces applied to the introducer housing 376 and prevent separation of the first and second housing members 378, 380 thereof.

As shown in FIGS. 25 and 26, a bottom joining surface 382, 384 is provided on each of first housing member 378 and second housing member 380, on the inner surfaces 320, 326 thereof, with the bottom joining surfaces 382, 384 being where the first housing member 378 and the second housing member 380 meet flush to form the bottom surface 312 of introducer housing 376. Each of the first housing member bottom joining surface 382 and the second housing member bottom joining surface 384 includes thereon additional mating features—separate from snaps 338, 342, 372—that aid in maintaining alignment and engagement between the first and second housing members 378, 380 when a twisting force is applied to the introducer housing 376. In the illustrated embodiment, the mating features are provided as a plurality of detents 386 formed in the bottom joining surface 382 of first housing member 378 that are spaced apart between the proximal end 316 and the distal end 318, and a plurality of bump outs 388 formed along the bottom joining surface 384 of second housing member 380 that are spaced apart between the proximal end 322 and the distal end 324 so as to be aligned with the plurality of detents 386. The bump outs 388 on second housing member 380 are configured to interfit with the detents 386 when the first and second housing members 378, 380 are secured together (by snaps 338, 342, 372).

When one or more regions of the introducer housing 376 is exposed to a twisting motion, such as a clockwise twisting of the proximal end 306 while the distal end 308 remains held straight, torsional force is applied to the snaps 338, 342, 372 that may stress the snaps (i.e., stress the hook portion 348 and/or beam sections) and urge the snaps toward separating from their respective mating surface 344. Specifically, application of a clockwise twisting motion to the proximal end 306 of introducer housing 376 may urge the downward oriented hook portion 348 of lower snaps 338 upward and the distally oriented hook portion 348 of upper snap 372 upward and proximally forward, which could cause disengagement of the upper snap 372 from its mating surface 344 on second housing member 380.

However, the inclusion of the bump outs 388 and detents 386 on the bottom joining surfaces 382, 384 of the first and second housing members 378, 380, respectively, prohibits the lower snaps 338 from disengaging mating surfaces 344 on the second housing member 380 when the proximal end 306 of the introducer housing 376 is twisted relative to the distal end 308. That is, even in the event that one of upper snaps 372, 342 fails or becomes disengaged from its respective mating surface 344 upon twisting of the introducer housing 376, the mating between bump outs 388 and detents 386 along the bottom joining surfaces 382, 384 of the first and second housing members 378, 380 assists in maintaining engagement between the lower snaps 338 and the mating surfaces 344 on the second housing member 380. Accordingly, engagement between the first housing member 378 and the second housing member 380 is maintained when the introducer housing 376 is exposed to a twisting motion.

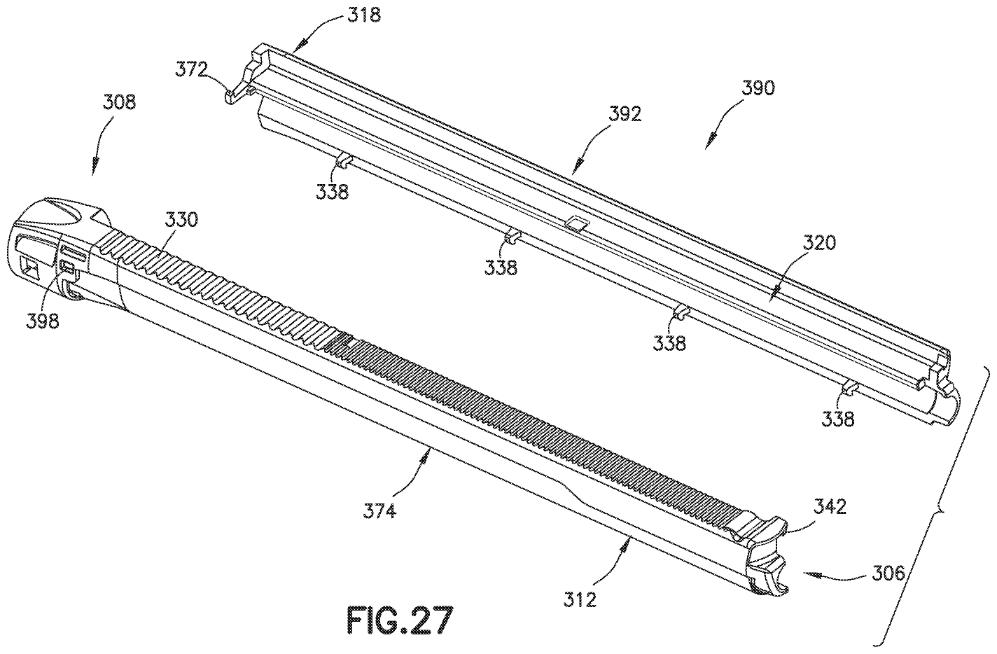

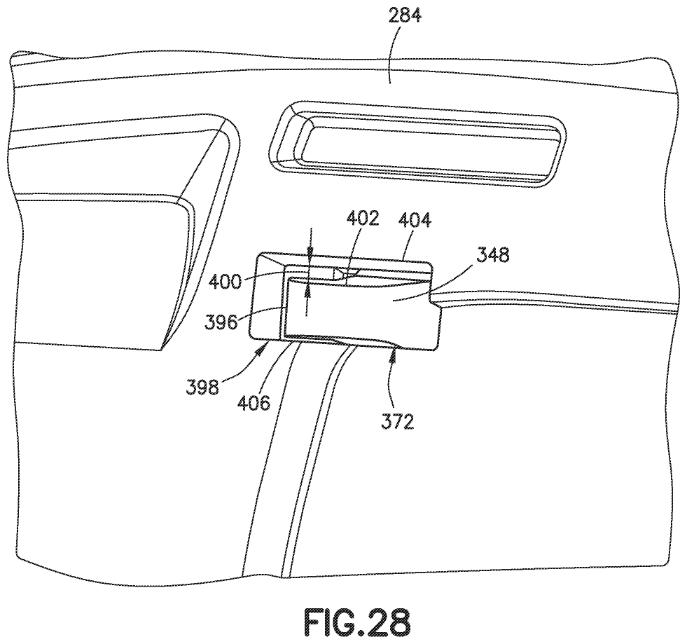

Referring now to FIGS. 27 and 28, another embodiment of an introducer housing 390 is provided that is resistant to torsional forces applied thereto. The arrangement and configurations of snaps on the introducer housing 390 of FIGS. 27 and 28 is identical to that of the introducer housings 366, 376 of FIGS. 23-24 and FIGS. 25-26 (i.e., lower snaps 338 and a second upper snap 342 with downward oriented hook portion 348s and a first upper snap 372 with a distally oriented hook portion 348), but movement of the upper snap 372 is restricted by the introducer housing 390 to counteract torsional forces applied to the introducer housing 390 and prevent separation of the first and second housing members 392, 394.

A more detailed view of the mating of upper snap 372 with a mating surface 344 on second housing member 394 is shown in FIG. 28. As seen therein the distally oriented hook portion 348 of upper snap 372 engages a mating surface 344 on second housing member 394, with an inner or front surface 396 of the hook portion 348 engaging the mating surface 344. A window 398 is formed in the second housing member 394 in an area adjacent the mating surface 344 such that, when the hook portion 348 is engaged with the mating surface 344, the hook portion 348 is received within the window 398 (i.e., a back surface of hook portion 348 extends partially out through the window 398) and is surrounded or encircled thereby. According to embodiments, the hook portion 348 is sized relative to the window 398 and/or positioned within the window 398 such that little or no gap 400 is present between an upper edge 402 of the hook portion 348 and an adjacent upper edge 404 of the window 398. In one embodiment, the hook portion 348 and window 398 are positioned relative to one another such that the hook portion 348 is vertically centered within the window 398, with the hook portion 348 sized relative to the window 398 such that the gap 400 has a width of 0.1 mm or less between the upper edge 402 of hook portion 348 and the upper edge 404 of window 398. In another embodiment, the hook portion 348 is positioned vertically off-center within the window 398 closer to the upper edge 404 of window 398 than a bottom edge 406 of window 398, with the hook portion 348 positioned such that a gap 400 of 0.1 mm or less is present between the upper edge 402 of hook portion 348 and the upper edge 404 of window 398. With a gap of 0.1 mm or less provided between the upper edge 402 of hook portion 348 and the upper edge 404 of window 398, vertical movement of the hook portion 348 along the mating surface 344 in the upward direction is restricted.

When one or more regions of the introducer housing 390 is exposed to a twisting motion, such as a clockwise twisting of the proximal end 306 while the distal end 308 remains held straight, torsional force is applied to the snaps 338, 342, 372 that may stress the snaps (i.e., stress the hook portion 348 and/or beam sections) and urge the snaps toward separating from their respective mating surface 344. Specifically, application of a clockwise twisting motion to the proximal end 306 of introducer housing 390 may urge the downward oriented hook portion 348 of lower snaps 338 upward and the distally oriented hook portion 348 of upper snap 372 upward and proximally forward, which could cause disengagement of the upper snap hook portion 348 from its mating surface 344 on second housing member 394.

However, by minimizing the size of the gap 400 present between the upper edge 402 of hook portion 348 of upper snap 372 and the upper edge 404 of window 398 within which it is received, an upward vertical movement of the hook portion 348 along the mating surface 344 is restricted when the proximal end 306 of the introducer housing 390 is twisted relative to the distal end 308. Restriction of the upward vertical movement of the hook portion 348 of upper snap 372 along its mating surface 344, in turn, restricts an associated proximal movement of the hook portion 348 of upper snap 372 that might cause it to disengage from its mating surface 344. Accordingly, engagement between the first housing member 392 and the second housing member 394 is maintained when the introducer housing 390 is exposed to a twisting motion.

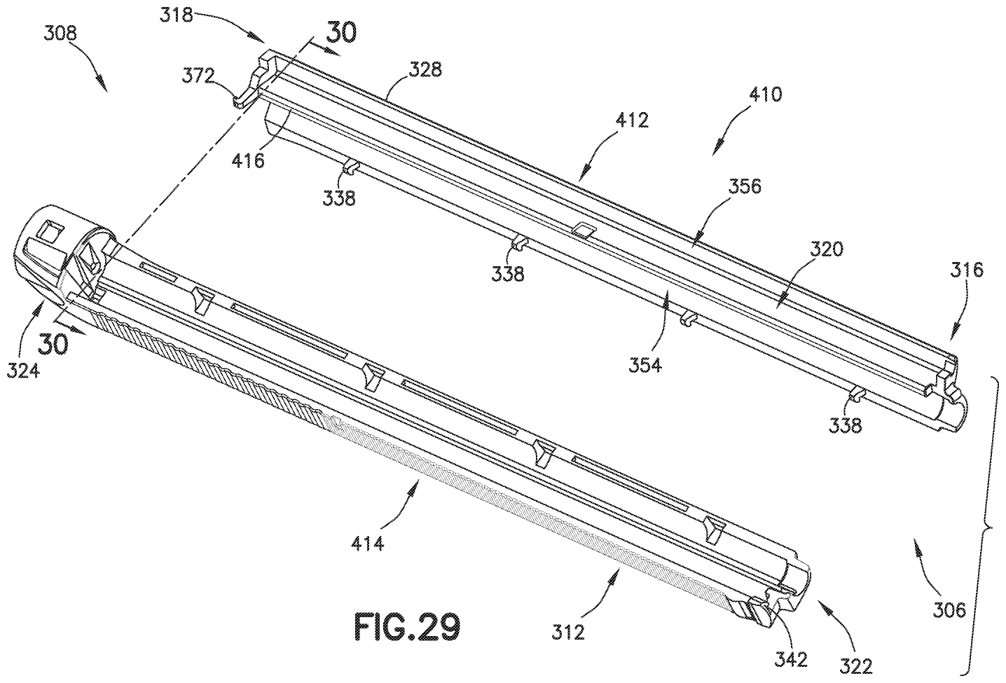

Referring now to FIGS. 29 and 30, another embodiment of an introducer housing 410 is provided that is resistant to torsional forces applied thereto. Again, the arrangement and configurations of snaps on the introducer housing 410 of FIGS. 29 and 30 is identical to that of each of the introducer housings 366, 376, 390 of FIGS. 23-24, FIGS. 25-26, and FIGS. 27-28 (i.e., lower snaps 338 and a second upper snap 342 with downward oriented hook portion 348s and a first upper snap 372 with a distally oriented hook portion 348), but additional alignment and mating features are provided in the introducer housing 410 to counteract torsional forces applied to the introducer housing 410 and prevent separation of the first and second housing members 412, 414 thereof.

As shown in FIGS. 29 and 30, the inner surface 320 of first housing member 412 includes a horizontally oriented ledge 416 extending from the proximal end 316 to the distal end 318, with the ledge 416 generally separating the lower portion 354 of inner surface 320 from an upper portion 356. A flange 418 on second housing member 414 (that forms the top surface 328 of second housing member 414) is formed such that, when the first housing member 412 is joined to the second housing member 414, a portion of the flange 418 overhangs the ledge 416, as best seen in FIG. 30.

When one or more regions of the introducer housing 410 is exposed to a twisting motion, such as a clockwise twisting of the proximal end 306 while the distal end 308 remains held straight, torsional force is applied to the snaps 338, 342, 372 that may stress the snaps (i.e., stress the hook portion 348 and/or beam sections) and urge the snaps toward separating from their respective mating surface 344. Specifically, application of a clockwise twisting motion to the proximal end 306 of introducer housing 410 may urge the downward oriented hook portion 348 of lower snaps 338 upward and the distally oriented hook portion 348 of upper snap 372 upward and proximally forward, which could cause disengagement of the upper snap hook portion 348 from its mating surface 344 on second housing member 414.

To prevent such disengagement of the lower snaps 338 and/or upper snap from their respective mating surfaces 344, one or more stop features 420 is provided on the bottom side of the flange 418 that inhibits vertical movement of the first housing member 412 relative to the second housing member 414. In one embodiment, the stop feature 420 is provided via the flange 418 having a vertical thickness along a length thereof that reduces the size of a vertical gap 422 between an underside abutment surface 424 of the flange 418 and the ledge 416. In another embodiment, the stop feature 420 is provided as one or more rib portions extending downward from the flange 418—at the proximal end 322 and/or distal end 324 of the second housing member 414—that reduces the size of the vertical gap 422 between an underside abutment surface 424 of the rib(s) and the ledge 416. According to embodiments, the stop feature 420 is configured so that the abutment surface 424 thereof is separated from the ledge 416 by a vertical gap 422 of 0.1 mm or less, including a zero gap (i.e., abutment surface 424 contacts ledge 416).

By minimizing the size of the gap 422 between the abutment surface 424 and the ledge 416, an upward vertical movement of the ledge 416 is restricted when the proximal end 306 of the introducer housing 410 is twisted relative to the distal end 308. Restriction of the upward vertical movement of the ledge 416, in turn, restricts an upward and associated proximal movement of the upper snap 372 that might cause it to disengage from its mating surface 344 and/or restricts an upward movement of the lower snaps 338 that might cause them to disengage from their respective mating surfaces 344. Accordingly, engagement between the first housing member 412 and the second housing member 414 is maintained when the introducer housing 410 is exposed to a twisting motion.

In any of the embodiments of introducer housings 300, 366, 376, 390, 410 in FIGS. 17-30, it is recognized that the structure of any of the snaps 338, 340, 342, 372 could be modified to further prevent breaking thereof that might occur responsive to twisting of the introducer housing. In some embodiments, a width, length or height of the hook portion 348 of one or more of the snaps 338, 340, 342, 372 (e.g., first upper snap 340, 372) may be increased to strengthen the hook portion 348, without affecting the ability of the hook to deflect during engaging thereof with an associated mating surface 344. In other embodiments, a width or height of the beam member 346 of one or more of the snaps 338, 340, 342, 372 (e.g., first upper snap 340, 372) may be increased to strengthen the beam member 346.

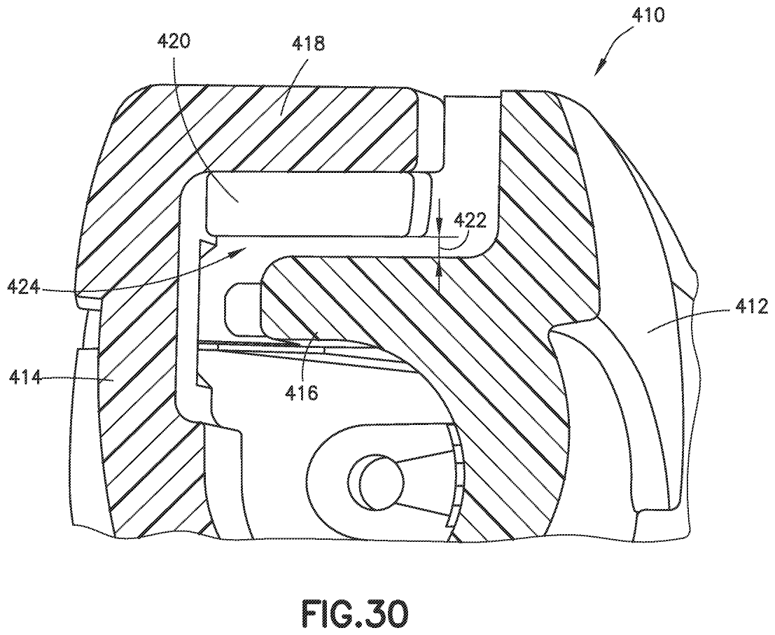

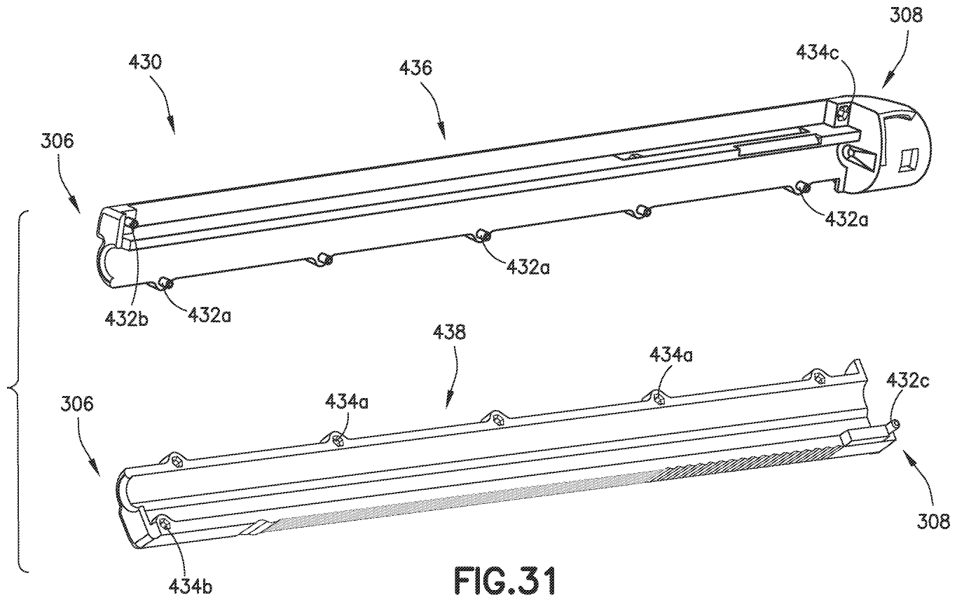

Referring now to FIGS. 31-34, another embodiment of an introducer housing 430 is provided that is resistant to torsional forces applied thereto. In the illustrated embodiment, the plurality of cantilevered snaps 338, 340, 342, 372 (and corresponding mating surfaces 344) provided on the introducer housings 300, 366, 376, 390, 410 of FIGS. 17-30 are replaced with a plurality of pegs 432 and a plurality of peg holes 434 joinable via a press fit to secure a first housing member 436 and a second housing member 438.

In one embodiment, a plurality of lower peg-peg hole pairs (i.e., lower pegs 432a and lower peg holes 434a) is provided on the first housing member 436 and the second housing member 438 arranged so as to be spaced apart between the proximal end 306 and the distal end 308 of the introducer housing 430. In the illustrated embodiment, lower pegs 432a are formed on first housing member 436 and lower peg holes 434a are formed in second housing member 438, but it is recognized that the lower pegs 432a could instead be formed on the second housing member 438 and the lower peg holes 434a formed in the first housing member 436, according to another embodiment. Additionally, a first upper peg-peg hole pair (i.e., first upper peg 432b and first upper peg hole 434b) is provided on the first housing member 436 and the second housing member 438 at the distal end 308 of introducer housing 430 and a second upper peg-peg hole pair (i.e., second upper peg 432c and second upper peg hole 434c) is provided on the first housing member 436 and the second housing member 438 at the proximal end 306 of the introducer housing 430. In the illustrated embodiment, the first upper peg 432b is formed on first housing member 436 and a first upper peg hole is formed in second housing member 438 while a second upper peg is formed on second housing member 438 and a second upper peg hole is formed on first housing member 436, but it is recognized that the arrangement of the upper pegs 432b, 432c and upper peg holes 434b, 434c could be switched, according to another embodiment.

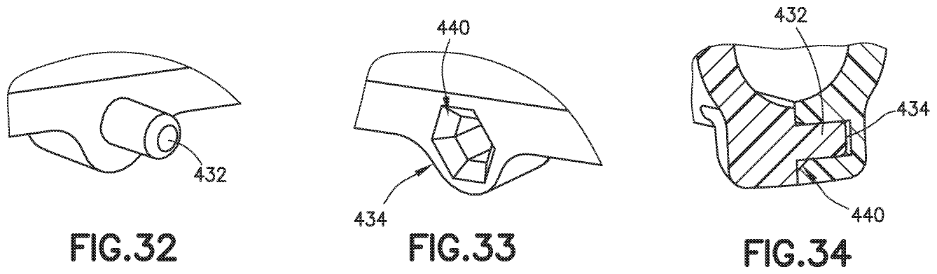

In some embodiments, the pegs 432 have a cross-sectional shape that is different from the cross-sectional shape of an opening of the peg holes 434, so as to reduce a stress of the press fit between the pegs 432 and the peg holes 434. In the example shown in FIGS. 32-34, the pegs 432 have a circular cross-section while the openings 440 of the peg holes 434 have a hexagonal shape, with it recognized that the pegs 432 and/or peg holes 434 could be configured to have various other suitable cross-sectional shapes.

The arrangement of the peg-peg hole pairs on the first and second housing members 436, 438 and the press fit engagement between the respective pegs 432 and peg holes 434 functions to secure the first and second housing members 436, 438 together, even when one or more regions of the introducer housing 430 is exposed to a twisting motion. That is, as compared to the cantilevered snaps 338, 340, 342, 372 on the introducer housings 300, 366, 376, 390, 410 as provided in FIGS. 17-30, the peg-peg hole pairs would not be subject to an “unhooking” or other disengagement that might occur responsive to the introducer housing 430 being exposed to a twisting motion. Accordingly, engagement between the first housing member 436 and the second housing member 438 is maintained via inclusion of the peg-peg hole pairs on the introducer housing 430.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A blood draw device for use with a peripheral intravenous catheter (PIVC) comprising:
a catheter;
an introducer housing having a proximal end and a distal end, and a top surface and a bottom surface, the housing having a first housing member and a second housing member each extending between the proximal end and the distal end, with the first housing member and the second housing member coupled together via a plurality of snaps formed thereon to define an inner volume configured to movably receive the catheter; and
an actuator movably coupled to the introducer housing, the actuator configured to move relative to the introducer housing to move a distal end of the catheter from a position within the introducer housing to a position outside the introducer housing;
wherein the plurality of snaps comprises:
a plurality of lower snaps formed on the first housing member and spaced apart between the proximal end and the distal end, each of the plurality of lower snaps extending out from the first housing member from a snap proximal end to a snap distal end, the snap distal end of each of the plurality of lower snaps comprising a hook that is oriented downward toward the bottom surface;
a first upper snap formed on the first housing member at the distal end, the first upper snap extending out from the first housing member from a snap proximal end to a snap distal end, the snap distal end of the first upper snap comprising a hook that is oriented upward toward the top surface; and
a second upper snap formed on the second housing member at the proximal end, the second upper snap extending out from the second housing member from a snap proximal end to a snap distal end, the snap distal end of the second upper snap comprising a hook that is oriented downward toward the bottom surface.

2. The blood draw device of claim 1, wherein the first upper snap is driven further into a mating surface of the second housing member responsive to a twisting of the proximal end of the introducer housing relative to the distal end drives, to prevent separation of the first housing member from the second housing member.

3. The blood draw device of claim 2, further comprising an alignment feature formed on the first housing member on the distal end thereof, below the first upper snap, the alignment feature engaging another mating feature on the second housing member to prevent disengaging of the first upper snap from the mating surface on the second housing member.

4. The blood draw device of claim 3, wherein the alignment feature comprises another lower snap extending out from the first housing member from a snap proximal end to a snap distal end, the snap distal end of the another lower snap comprising a hook that is oriented downward toward the bottom surface.

5. The blood draw device of claim 1, wherein the first housing member forms a portion of the top surface and the second housing member forms another portion of the top surface, the portion of the top surface separated from the another portion of the top surface to define a groove that extends generally from the proximal end to the distal end, and wherein a portion of the actuator is engaged with the groove to enable movement of the actuator relative to the introducer housing.

6. A blood draw device for use with a peripheral intravenous catheter (PIVC) comprising:
a catheter;
an introducer housing having a proximal end and a distal end, and a top surface and a bottom surface, the introducer housing having a first housing member and a second housing member each extending between the proximal end and the distal end, with the first housing member and the second housing member coupled together via a plurality of snaps formed thereon to define an inner volume configured to movably receive the catheter;
an actuator movably coupled to the introducer housing, the actuator configured to move relative to the introducer housing to move a distal end of the catheter from a position within the introducer housing to a position outside the introducer housing;
wherein the plurality of snaps comprises:
a plurality of lower snaps formed on the first housing member and spaced apart between the proximal end and the distal end, each of the plurality of lower snaps extending out from the first housing member from a snap proximal end to a snap distal end, the snap distal end of each of the plurality of lower snaps comprising a hook that is oriented downward toward the bottom surface;
a first upper snap formed on the first housing member at the distal end, the first upper snap extending out from the first housing member from a snap proximal end to a snap distal end, the snap distal end of the first upper snap comprising a hook that is oriented toward the distal end of the introducer housing; and
a second upper snap formed on the second housing member at the proximal end, the second upper snap extending out from the second housing member from a snap proximal end to a snap distal end, the snap distal end of the second upper snap comprising a hook that is oriented downward toward the bottom surface; and
wherein the second housing member comprises a snap stop positioned at the distal end, the snap stop extending out from the second housing member so as to be positioned proximally behind the first upper snap, adjacent to a back side of the hook thereof.

7. The blood draw device of claim 6, wherein the snap stop prohibits proximal movement of the first upper snap relative to the second housing member, thereby preventing the first upper snap from disengaging a mating surface of the second housing member.

8. The blood draw device of claim 6, wherein the snap stop comprises a cap piece inserted into the second housing member.

9. A blood draw device for use with a peripheral intravenous catheter (PIVC) comprising:
a catheter;
an introducer housing having a proximal end and a distal end, and a top surface and a bottom surface, the introducer housing having a first housing member and a second housing member each extending between the proximal end and the distal end, with the first housing member and the second housing member coupled together via a plurality of snaps formed thereon to define an inner volume configured to movably receive the catheter;
an actuator movably coupled to the introducer housing, the actuator configured to move relative to the introducer housing to move a distal end of the catheter from a position within the introducer housing to a position outside the introducer housing;
wherein the first housing member comprises a bottom joining surface extending from the proximal end to the distal end, the bottom joining surface comprising a plurality of detents formed therein that are spaced apart between the proximal end and the distal end; and
wherein the second housing member comprises another bottom joining surface that adjoins with the bottom joining surface of the first housing member, the another bottom joining surface comprising a plurality of bump outs formed thereon that are spaced apart between the proximal end and the distal end, so as to be aligned with the plurality of detents and interfit therewith.

10. The blood draw device of claim 9, wherein the plurality of snaps comprises:
a plurality of lower snaps formed on the first housing member and spaced apart along the bottom joining surface, each of the plurality of lower snaps extending out from the first housing member from a snap proximal end to a snap distal end, the snap distal end of each of the plurality of lower snaps comprising a hook that is oriented downward toward the bottom surface;
a first upper snap formed on the first housing member at the distal end, the first upper snap extending out from the first housing member from a snap proximal end to a snap distal end, the snap distal end of the first upper snap comprising a hook that is oriented toward the distal end of the introducer housing; and
a second upper snap formed on the second housing member at the proximal end, the second upper snap extending out from the second housing member from a snap proximal end to a snap distal end, the snap distal end of the second upper snap comprising a hook that is oriented downward toward the bottom surface.

11. The blood draw device of claim 9, wherein interfitting of the plurality of bump outs with the plurality of detents prevents the plurality of lower snaps from disengaging mating surfaces on the second housing member when the proximal end of the introducer housing is twisted relative to the distal end, to prevent separation of the first housing member from the second housing member.

12. A blood draw device for use with a peripheral intravenous catheter (PIVC) comprising:
a catheter;
an introducer housing having a proximal end and a distal end, and a top surface and a bottom surface, the introducer housing having a first housing member and a second housing member each extending between the proximal end and the distal end, with the first housing member and the second housing member coupled together via a plurality of snaps formed thereon to define an inner volume configured to movably receive the catheter;
an actuator movably coupled to the introducer housing, the actuator configured to move relative to the introducer housing to move a distal end of the catheter from a position within the introducer housing to a position outside the introducer housing;
wherein the plurality of snaps comprises:
a plurality of lower snaps formed on the first housing member and spaced apart between the proximal end and the distal end, each of the plurality of lower snaps extending out from the first housing member from a snap proximal end to a snap distal end, the snap distal end of each of the plurality of lower snaps comprising a hook that is oriented downward toward the bottom surface;
a first upper snap formed on the first housing member at the distal end, the first upper snap extending out from the first housing member from a snap proximal end to a snap distal end, the snap distal end of the first upper snap comprising a hook that is oriented toward the distal end of the introducer housing; and
a second upper snap formed on the second housing member at the proximal end, the second upper snap extending out from the second housing member from a snap proximal end to a snap distal end, the snap distal end of the second upper snap comprising a hook that is oriented downward toward the bottom surface; and
wherein the second housing member comprises:
a mating surface engaged by the hook of the first upper snap; and
a window formed in the second housing member adjacent the mating surface, the window encircling the hook of the first upper snap;
wherein the window is sized to restrict vertical movement of the hook of the first upper snap.

13. The blood draw device of claim 12, wherein a vertical gap between an edge of the window and a side surface of the hook of the first upper snap is 0.1 mm or less.

14. A blood draw device for use with a peripheral intravenous catheter (PIVC) comprising:
a catheter;
an introducer housing having a proximal end and a distal end, and a top surface and a bottom surface, the introducer housing having a first housing member and a second housing member each extending between the proximal end and the distal end, with the first housing member and the second housing member coupled together to define an inner volume configured to movably receive the catheter;
an actuator movably coupled to the introducer housing, the actuator configured to move relative to the introducer housing to move a distal end of the catheter from a position within the introducer housing to a position outside the introducer housing;
wherein the first housing member and the second housing member comprise a plurality of pegs and a plurality of peg holes joinable to secure the first housing member and the second housing member, the plurality of pegs being press fit into the plurality of peg holes.

15. The blood draw device of claim 14, wherein the plurality of pegs and the plurality of peg holes comprises:
a plurality of lower peg-peg hole pairs provided on the first housing member and the second housing member and arranged so as to be spaced apart between the proximal end and the distal end;
a first upper peg-peg hole pair provided on the first housing member and the second housing member at the distal end; and
a second upper peg-peg hole pair provided on the first housing member and the second housing member at the proximal end.

16. The blood draw device of claim 14, where each of the plurality of pegs has a first cross-sectional shape and each of the plurality of peg holes has an opening with a second cross-sectional shape different from the first cross-sectional shape, to reduce a stress of the press fit between the pegs and the peg holes.

17. A blood draw device for use with a peripheral intravenous catheter (PIVC) comprising:
a catheter;
an introducer housing having a proximal end and a distal end, and a top surface and a bottom surface, the introducer housing having a first housing member and a second housing member each extending between the proximal end and the distal end, with the first housing member and the second housing member coupled together via a plurality of snaps formed thereon to define an inner volume configured to movably receive the catheter;
an actuator movably coupled to the introducer housing, the actuator configured to move relative to the introducer housing to move a distal end of the catheter from a position within the introducer housing to a position outside the introducer housing;
wherein the plurality of snaps comprises:
a plurality of lower snaps formed on the first housing member and spaced apart between the proximal end and the distal end, each of the plurality of lower snaps extending out from the first housing member from a snap proximal end to a snap distal end, the snap distal end of each of the plurality of lower snaps comprising a hook that is oriented downward toward the bottom surface; and
a first upper snap formed on the first housing member at the distal end, the first upper snap extending out from the first housing member from a snap proximal end to a snap distal end, the snap distal end of the first upper snap comprising a hook that is oriented toward the distal end of the introducer housing;
a second upper snap formed on the second housing member at the proximal end, the second upper snap extending out from the second housing member from a snap proximal end to a snap distal end, the snap distal end of the second upper snap comprising a hook that is oriented downward toward the bottom surface; and
wherein the first housing member comprises a horizontally oriented ledge extending from the proximal end to the distal end; and
wherein the second housing member comprises a stop feature positioned on an underside of the top surface, the stop feature having an abutment surface facing the horizontally oriented ledge and arranged parallel thereto, with the abutment surface separated from the horizontally oriented ledge by a distance of 0.1 mm or less.

18. The blood draw device of claim 17, wherein the stop feature inhibits vertical movement of the first housing member relative to the second housing member responsive to a twisting of the proximal end of the introducer housing relative to the distal end, thereby preventing a corresponding proximal movement of the first upper snap off of a mating surface of the second housing member.

19. The blood draw device of claim 17, wherein the stop feature comprises a rib positioned at the proximal end and/or the distal end of the introducer housing.
